(19) United States
(12) Patent Application Publication
(10) Pub. No.: US 2011/0257146 A1
(43) Pub. Date: Oct. 20, 2011

(54) Method of Treating Kcnq Related Disorders Using Organozinc Compounds

(75) Inventors: Min Li, Lutherville, MD (US); Haiyan Sun, Chandler, AZ (US); Qiaojie Xiong, East Setauket, NY (US)

(73) Assignee: The johns Hopkins University, Baltimore, MD (US)

(21) Appl. No.: 12/223,136

(22) PCT Filed: Jan. 25, 2007
(86) PCT No.: PCT/US07/02116

§ 371 Date: Jul. 5, 2011

Related U.S. Application Data
(60) Provisional application No. 60/762,074, filed on Jan. 25, 2006.

(51) Int. Cl. A61K 31/555; A61K 31/315; G01N 33/53; A61K 31/27

(52) U.S. Cl. 514/187; 514/188; 514/184; 514/478; 514/494; 436/501

(57) ABSTRACT

The instant invention describes methods of treating KCNQ related diseases and disorders using organozinc compounds. In certain embodiments, pain is treated using Zinc Pyrithione.

### RELATED APPLICATION

This application claims the benefit of U.S. provisional patent application Ser. No. 60/762,074, filed Jan. 25, 2006, the disclosure of which is incorporated herein in its entirety by this reference.

### BACKGROUND

Ion channels are cellular proteins that regulate the flow of ions, including calcium, potassium, sodium and chloride, into and out of cells. These channels are present in all human cells and affect such processes as nerve transmission, muscle contraction and cellular secretion. Among the ion channels, potassium channels are the most ubiquitous and diverse, being found in a variety of animal cells such as nervous, muscular, glandular, immune, reproductive, and epithelial tissue. These channels allow the flow of potassium in and/or out of the cell under certain conditions. For example, the outward flow of potassium ions upon opening of these channels makes the interior of the cell more negative, counteracting depolarizing voltages applied to the cell. These channels are regulated, e.g., by calcium sensitivity, voltage-gating, second messengers, extracellular ligands, and ATP-sensitivity.

Potassium channels are involved in a number of physiological processes, including regulation of heartbeat, dilation of arteries, release of insulin, excitability of nerve cells, and regulation of renal electrolyte transport. Potassium channels are thus found in a wide variety of animal cells such as nervous, muscular, glandular, immune, reproductive, and epithelial tissue. These channels allow the flow of potassium in and/or out of the cell under certain conditions. For, example, the outward flow of potassium ions upon opening of these channels makes the interior of the cell more negative, counteracting depolarizing voltages applied to the cell. These channels are regulated, e.g., by calcium sensitivity, voltage-gating, second messengers, extracellular ligands, and ATP-sensitivity.

Potassium channels are made by alpha subunits that fall into at least 8 families, based on predicted structural and functional similarities (Wei et al., Neuropharmacology 35(7):805-829 (1997)). Three of these families (Kv, Eag-related, and KQT, now referred to as KCNQ) share a common motif of six transmembrane domains and are primarily gated by voltage. Two other families, CNG and SK/IK, also contain this motif but are gated by cyclic nucleotides and calcium, respectively. The three other families of potassium channel alpha subunits have distinct patterns of transmembrane domains. Slo family potassium channels, or BK channels, have seven transmembrane domains (Meera et al., Proc. Natl. Acad. Sci. U.S.A. 94(25):14066-71 (1997)) and are gated by both voltage and calcium or pH (Schreiber et al., J. Biol. Chem. 273:3509-16 (1998)). Another family, the inward rectifier potassium channels (Kir), belong to a structural family containing two transmembrane domains, and an eighth functionally diverse family (TP, or “two-pore”) contains two tandem repeats of this inward rectifier motif.

Potassium channels are typically formed by four alpha subunits, and can be homomeric (made of identical alpha subunits) or heteromeric (made of two or more distinct types of alpha subunits). In addition, potassium channels have often been found to contain additional, structurally distinct auxiliary, or beta, subunits (e.g., Kv, Slo, and KCNQ potassium channel families). These beta subunits do not form potassium channels themselves, but instead they act as auxiliary subunits to modify the functional properties of channels formed by alpha subunits. For example, the Kv beta subunits are cytoplasmic and are known to increase the surface expression of Kv channels and/or modify inactivation kinetics of the channel (Heinemann et al., J. Physiol. 493:625-633 (1996); Shi et al., Neuron 16(4):843-852 (1996)). In another example, the KCNQ family beta subunit, minK, primarily changes activation kinetics (Sanguinetti et al., Nature 384:80-83 (1996)).

Certain members of the Kv family of potassium channels were recently renamed (see Biervert et al., Science 279:403-406 (1998)). KvLQT1 was re-named KCNQ1, and the KvLQT1-related channels (KvLR1 and KvLR2) were renamed KCNQ2 and KCNQ3, respectively. More recently, a fourth member of the KCNQ subfamily was identified (KCNQ4) as a channel expressed in sensory outer hair cells (Kubisch et al., Cell 96(3): 437-446 (1999)).

The KCNQ family of potassium channels was first identified in humans on the basis of inherited mutations that cause the Long QT syndrome (Wang et al., Nat. Genet. 12:17-23 (1996)). The mutations were found in a potassium channel, KVLQT1, now known as KCNQ1, that was structurally distinct from previously cloned voltage-gated potassium channels. More recently, it has been discovered that KCNQ1 represents a larger family of structurally similar voltage-gated potassium channels. Three more members of this novel voltage-gated potassium channel family, KCNQ2, KCNQ3, and KCNQ4, have been cloned from humans (Charlier et al., Nat. Genet. 18:53-55; Biervert et al., Science 279:403-406 (1998); Singh et al., Nat. Genet. 18:25-29 (1998); Yang et al., J. Biol. Chem. 273:19419-19423 (1998); and Kubisch et al., Cell 96:437-446 (1999)). Mutations in each member of the KCNQ gene family have been linked to inherited human disease. For example, KCNQ1 has been linked to the Long QT syndrome, as described above. KCNQ2 and KCNQ3 have been linked to certain forms of epilepsy (Charlier et al., Nat. Genet. 18:53-55; Biervert et al., Science 279: 403-406 (1998); and Singh et al., Nat. Genet. 18:25-29 (1998). KCNQ4 has been linked to deafness (Kubisch et al., Cell 96:437-446 (1999)).

Zinc plays a critical role in cellular biology, and is involved in virtually every important cellular process such as transcription, translation, ion transport, and others (O'Halloran, T. V. (1993) Science 261:715-725; Cousins, R. J. (1994) Annu. Rev. Nutr. 14:449-469; Harrison, N. L. et al. (1994) Neuropharmacology 33:935-952; Berg, J. M. et al. (1996) Science 271:1081-1085). The involvement of cellular zinc in apoptosis has been recognized for close to twenty years (Sunderman, F. W., Jr. (1995) Ann. Clin. Lab. Sci. 25:134-142; Fraker, P. J. et al. (1997) Proc. Soc. Exp. Biol. Med. 215:229-236.). However, the full nature of this involvement is not fully understood. Apoptosis is a form of programmed cell death normally activated under physiological conditions, such as involution in tissue remodelling during morphogenesis, and several immunological processes. The apoptotic process is characterized by cell shrinkage, chromatin condensation, and internucleosomal degradation of the cell's DNA (Verhaegen et al. (1995) Biochem. Pharmacol. 50(7):1021-1029).

Zinc-pyrithione, commercially available from Sigma, is the active ingredient in the anti-dandruff shampoo Head & Shoulders® (U.S. Pat. Nos. 3,236,733, and 3,281,366, both 1966), as well as a number of other topical skin treatment formulations. It is a fungicide and bactericide at high concentrations. It is highly lipophilic and therefore penetrates membranes easily. This permits zinc pyrithione to transport zinc across cell membranes, thereby conferring on this compound (i.e. zinc pyrithione) the properties of a zinc ionophore. The anti-apoptotic effect of zinc pyrithione was first observed in vitro by Zalewski and coworkers, who showed that micromolar concentrations of this compound protected lymphocytic leukemia cells against colchicine-induced apoptosis (Giannakis, C., et al. (1991) Biochem. Biophys. Res. Commun. 181:915-920). The rationale for the use of this zinc ionophore was to facilitate the transport of Zn²⁺ into the target cells. This is necessitated by the fact that all eukaryotic cells strictly regulate the membrane transport of Zn²⁺, making it very difficult to modulate the intracellular concentration and distribution of Zn²⁺. Zalewski's group has since published a number of other studies, all of them in vitro, confirming the ability of micromolar concentrations of zinc-pyrithione to rapidly transport Zn²⁺ into cells and to thereby prevent apoptosis (Zalewski, P. D., et al. (1994) supra; Zalewski, P. D., et al. (1993) Biochem. J. 296:403-408). One confirmatory study, also in vitro, has been published from another laboratory (Tempel, K.-H. et al., (1993) Arch. Toxicol. 67:318-324).

### BRIEF SUMMARY OF THE INVENTION

The present invention relates to the use of organozinc compounds in methods of treating KCNQ related disorders, by increasing ion flow in KCNQ polypeptide channels and activating KCNQ currents by opening the channels via chemical and electrical interactions.

In one aspect, the invention provides a method of treating a KCNQ related disorder in a subject, comprising administering to said subject, an effective amount of a KCNQ polypeptide channel activity modulator, such that said subject is treated for said disorder; wherein the modulator of a KCNQ polypeptide channel is an organozinc compound.

In another aspect, the invention provides a method of treating a KCNQ related disorder in a subject, wherein the subject has been identified as in need of treatment for a KCNQ related disorder, comprising administering to said subject in need thereof, an effective amount of a KCNQ polypeptide channel activity modulator, such that said subject is treated for said disorder, wherein the modulator of a KCNQ polypeptide channel is an organozinc compound.

The methods of the invention are directed towards the treatment of pain using organozinc compounds, such as zinc pyrithione (ZnPy).

In another aspect, the invention provides a method of protecting against the effects of pain comprising, administering to a subject in need thereof, a pharmaceutically effective amount of a KCNQ polypeptide channel activity modulator; wherein the modulator of a KCNQ polypeptide channel is an organozinc compound.

In still another aspect, the invention provides a method of modulating the activity of a potassium channel in a subject, comprising contacting a potassium channel with an organozinc compound. In another aspect, the invention provides a method of modulating the activity of a potassium channel in a subject, comprising administering to said subject an effective amount of an organozinc compound.

In yet another aspect, the invention provides a method of treating a KCNQ mutation disorder in a subject, comprising administering to the subject, an effective amount of a KCNQ polypeptide channel activity potentiator, such that said subject is treated for said disorder; wherein the potentiator of a KCNQ polypeptide channel is an organozinc compound.

In certain aspects, the invention provides for a method for identifying a compound which modulates the activity of a KCNQ polypeptide channel, the method comprising: a) contacting a KCNQ polypeptide channel with a compound under conditions suitable for modulation of the activity of the KCNQ polypeptide channel; and b) detecting modulation of the activity of the KCNQ polypeptide channel by the compound; wherein the compound chemically interacts with the KCNQ polypeptide channel.

### DETAILED DESCRIPTION

### Brief Description of the Drawings

FIG. 1. ZnPy (ZnPy) potentiated potassium currents induced by KCNQ homomultimers. A. Whole-cell currents of CHO cells transfected individually with KCNQ1 to KCNQ5 were recorded in the absence (left panels) and presence (right panels) of 10 μM ZnPy. Holding potential (Vₕ) was −80 mV, in 10 mV incremental voltage steps from −70 mV to +50 mV (insert). Scale bars are as indicated. B. A histogram plotting the ZnPy effect on KCNQ currents at both −30 mV (open box) and +50 mV (filled box). The current amplitude is shown as normalized current (I_{ZnPy}/I_{control}). The number of folds of current increase is indicated above each column (n≧4).

FIG. 2. Combinatorial effects on KCNQ2 current by potassium channel blockers and ZnPy. Representative time course recordings of KCNQ2 current at +50 mV are shown. The time periods of external application of 20 mM tetraethylammonium (TEA) (panel A) or 30 μM linopirdine (panel B) and overlapping applications of ZnPy are as shown (n≧4).

FIG. 3. Complexed form of ZnPy is required for potentiation. A. Representative time course recordings of KCNQ2 current at +50 mV are shown. The overlapping time periods of external application of 20 μM sodium pyrithione (NaPy), 10 μM ZnSO₄, and 100 μM 4-(2)-(pyridylazo) resorcinol (PAR) are as shown. B. The non-overlapping time periods of external applications are shown. The first application is a mixture of 10 μM ZnSO₄ and 20 μM pyrithione (Py). The cell was then treated with 10 μM ZnPy upon the removal of the ZnSO₄ and pyrithione. C. Histogram summarizing the folds of potentiation at +50 mV by zinc ionophores including zinc diethyldithiocarbamate (DEDTC), 5,7-diiodo-8-hydroxyquinoline (DIQ) and (±)-α-tocopherol (VE) (n≧4, * p<0.001).

FIG. 4. Stoichiometric preference of Zn²⁺ and pyrithione. Structure of ZnPy is as shown according to the crystallographic studies. KCNQ2 current sensitivity was tested in the presence of mixtures of ZnSO₄ and NaPy at the indicated ratios. KCNQ2 potentiation is shown as normalized current obtained at +50 mV (n≧4).

FIG. 5. Biophysical effects on KCNQ channels by ZnPy. A-C. Conductance-voltage (G-V) curves of KCNQ2 homomultimers (A). KCNQ3 homomers (B) and KCNQ2/3 heteromers (C) in the absence (square) or presence (circle) of 10 μM ZnPy (n≧4, p<0.001). Conductance at each depolarized voltage (from −70 mV to +50 mV) was normalized to the conductance at +50 mV (Gₘₐₓ) in the absence of ZnPy. Dashed lines are fit curves for KCNQ2 (A) and KCNQ2/3 (C) in the presence of ZnPy after recalling the Gₘₐₓ to 1. D. Midpoint of activation voltage shifts of KCNQ2 (circle) and KCNQ2/3 (square) induced by ZnPy at different concentrations (n≧4). Curves shown are the fit of concentration-response curves to the mean values, with half maximal effective concentrations (EC₅₀) of 1.5 μM for KCNQ2 alone and 2.4 μM for KCNQ2/3 heteromultimers.

FIG. 6. ZnPy effects on KCNQ2 and KCNQ2/3 deactivation. A and B, left panel shows the scaled tail currents from full traces (insert). Gray lines are the currents recorded in the control condition, and black lines are in the presence of 10 μM ZnPy. Vₕ was −80 mV, stepped to +50 mV, followed by −120 mV hyperpolarization. Right panel shows the deactivation time constants in the absence and presence of 10 μM ZnPy (n≧4, * p<0.001).

FIG. 7. ZnPy increases the open channel probability (Pₒ) of KCNQ2 channel. A. Left panel, the single channel signals from outside-out patches clamped at 0 mV in the absence and presence of 10 μM ZnPy, or 50 μM linopirdine. Right panel, all-point amplitude histograms for the sweeps shown in left. The fitted unitary current amplitudes and Pₒ at 0 mV were: 0.51 pA and 0.13 (control), 0.52 pA and 0.77 (ZnPy), 0.52 pA and 0.17 (wash), respectively. B. Histograms show the Pₒ values at 0 mV in the absence and presence of ZnPy (n=5, *p<0.001). C. Histograms show the single channel conductance (γ) in the absence and presence of ZnPy (n=5).

FIG. 8. Differential modulatory sites on KCNQ channel by ZnPy and retigabine. A. Left Panel. Representative KCNQ2 current was recorded with intracellular pipet solution supplemented with 10 μM ZnPy. The overlapping external application of ZnPy is as shown. Right Panel. G-V curves at different indicated recording times (0 to 12 minutes) upon establishing stable seal are shown. The intracellular perfusion (with or without EGTA) was first calibrated by using compatible molecular weight dye. B. A histogram shows the comparison of ZnPy and retigabine (RTG) effects on KCNQ2 wild type and a KCNQ2 (W236L) mutant 2.5 μM ZnPy or 10 μM RTG was applied separately to KCNQ2 wildtype (wt) or W236L. The levels of current potentiation at +50 mV and −30 mV were calculated by I_{drug}/I_{control}, and shown as indicated (n≧4, *p<0.001).

FIG. 9. KCNQ2 point mutants selectively lose ZnPy sensitivity. A. Histogram shows the current (+50 mV) potentiation effect of ZnPy on KCNQ2 S5-S6 point mutants (n≧3). Each mutation site was indicated based on the predicted transmembrane regions. Dashed line indicates the potentiation level of wt KCNQ2 channel in the absence of ZnPy. B. Right panel shows the activation curves of KCNQ2(A306T) (blue) in the presence and absence of ZnPy; left panel shows the activation curves of KCNQ2(L249A/L275A) (red) in the presence and absence of ZnPy (n≧4, p>0.1). C. Modeling of the S5-pore-S6 region of KCNQ2.

FIG. 10. ZnPy potentiation of native M-current. A. M current recorded from an isolated rat dorsal root ganglion neuron, Vₕ was −60 mV, stepped to −20 mV, followed by a step to −50 mV, and stepped back to −20 mV. B. Histograms show M-current amplitudes in the presence and absence of 10 μM ZnPy (n=10, * p<0.001). M-current amplitudes were measured by fitting the I_{M} relaxation with an exponential equation. The recording bath solution includes 1 μM TTX, 0.2 mM TEA, 1 mM 4-AP, 50 μM CNQX, and 10 μM bicuculline. C. Membrane potential of an isolated hippocampal neuron was monitored when 10 μM ZnPy was applied in bath solution followed by the addition of 15 μM linopirdine. D. Action potentials elicited by 200 pA current injection to a patched hippocampal neuron. The ZnPy treatments are as indicated. E. Histogram shows the numbers of elicited action potentials before and after 10 μM ZnPy application (n=4, *p<0.001).

FIG. 11. ZnPy effects on BFNC mutant channels expressed in CHO cells. A. whole-cell currents of KCNQ2(R207W), KCNQ2(Y284C), and KCNQ2(A306T) were recorded in the absence (left) and presence (right) of 10 μM ZnPy, Vₕ was −80 mV, in 10 mV voltage steps from −70 mV to +50 mV. B. a histogram plotting potentiation effects on KCNQ2 wild type (wt) and BFNC mutants (R207W, Y284C and A3067) by ZnPy (filled box) and retigabine (open box) (n≧4, * p<0.001, ** p<0.05).

### DEFINITIONS

In order that the invention may be more readily understood, certain terms are first defined and collected here for convenience.

As used herein, the term “treating” pain encompasses preventing, ameliorating, mitigating and/or managing pain and/or conditions that may cause pain, such as inflammation. As used herein, “inhibiting” pain or inflammation encompasses preventing, reducing and halting progression of same.

The term “pain” refers to all categories of pain, including pain that is described in terms of stimulus or nerve response, e.g., somatic pain (normal nerve response to a noxious stimulus) and neuropathic pain (abnormal response of a injured or altered sensory pathway, often without clear noxious input); pain that is categorized temporally, e.g., chronic pain and acute pain; pain that is categorized in terms of its severity, e.g., mild, moderate, or severe; and pain that is a symptom or a result of a disease state or syndrome, e.g., inflammatory pain, cancer pain, AIDS pain, arthropathy, migraine, trigeminal neuralgia, cardiac ischaemia, and diabetic neuropathy (see, e.g., Harrison's Principles of Internal Medicine, pp. 93-98 (Wilson et al., eds., 12th ed. 1991); Williams et al., J. of Medicinal Chem. 42:1481-1485 (1999), herein each incorporated by reference in their entirety).

“Somatic” pain, as described above, refers to a normal nerve response to a noxious stimulus such as injury or illness, e.g., trauma, burn, infection, inflammation, or disease process such as cancer, and includes both cutaneous pain (e.g., skin, muscle or joint derived) and visceral pain (e.g., organ derived).

“Neuropathic” pain, as described above, refers to pain resulting from injury to or chronic changes in peripheral and/or central sensory pathways, where the pain often occurs or persists without an obvious noxious input.

The term “organozinc compound” herein refers to one or more organic functionalities, e.g., alkyl groups, aryl groups, alkoxy group, and the like, that bind, coordinate, or chelate to a zinc moiety.

A “zinc moiety” refers to zinc in any oxidation state or ionization state that is capable of directly binding, coordinating, or chelating to an organic group.

By “zinc ionophore” is meant a therapeutic compound complexed with zinc ions that is capable of carrying zinc ions across cell membranes.

“KCNQ polypeptide channel” refers to heteromeric or homomeric channels composed of at least one alpha subunit from the KCNQ polypeptide family.

“KCNQ potassium channel” refers to heteromeric or homomeric potassium channels composed of at least one alpha subunit from the KCNQ polypeptide family, as described below.

“KCNQ polypeptide” or “KCNQ subunit” refers to a polypeptide that is a subunit or monomer of a voltage-gated, KCNQ potassium channel, a member of the KCNQ gene family, or a member of the Kv superfamily of potassium channel monomers. When a KCNQ polypeptide, e.g., KCNQ 1, 2, 3, 4, or 5, is part of a KCNQ potassium channel, either a homomeric or heteromeric potassium channel, the channel has voltage-gated activity. The term KCNQ polypeptide therefore refers to polymorphic variants, alleles, mutants, physiological variants (including but not limited to alternative splicing, proteolytic cleavage), and interspecies homologs that: (1) have a sequence that has greater than about 60% amino acid sequence identity, preferably about 65, 70, 75, 80, 85, 90, or 95% amino acid sequence identity, to a KCNQ gene family member such as those described in Biervert et al., Science 279:403-406, Kubisch et al., Cell 96:437-446 (1999), Yang et al., J. Biol. Chem. 273:19419-19423 (1998); Wang et al., Nature Genet. 12:17 (1996); Wei et al., Neuropharmacol. 35:805 (1996); Singh et al., Nature Genet. 18:25 (1998); Charlier et al., Nature Genet. 18:53 (1998); WO 99/31232; and WO 99/07832 (herein each incorporated by reference in their entirety); (2) bind to antibodies, e.g., polyclonal antibodies, raised against an immunogen comprising a KCNQ gene family member polypeptide, as described above, and conservatively modified variants thereof; (3) specifically hybridize under stringent hybridization conditions to a sequence encoding a KCNQ gene family member polypeptide, as described above, and conservatively modified variants thereof; or (4) are amplified by primers that specifically hybridize under stringent hybridization conditions a sequence encoding a KCNQ gene family polypeptide, as described above.

KCNQ potassium channels, KCNQ polynucleotides, and KCNQ nucleic acids are identified, isolated, expressed, purified, and expressed in recombinant cells according to methods well known to those of skill in the art.

The term “KCNQ related disorder” refers to disorders that are caused by the mediation or modulation of a KCNQ polypeptide channel.

“Inhibitors,” “activators” “openers,” or “modulators” of voltage-gated potassium channels comprising a KCNQ subunit refer to inhibitory, activating, or modulatory molecules. In certain instances, such inhibitors, activators, or modulators are identified using in vitro and in vivo assays for KCNQ channel function. In particular, inhibitors, activators, and modulators refer to compounds that increase KCNQ channel function, thereby reducing pain in a subject, as assayed using a formalin algesia test or a hotplate test in vivo, or thereby reducing anxiety in a subject, as assayed using a Geller conflict test. Inhibitors are compounds that decrease, block, prevent, delay activation, inactivate, desensitize, or down regulate the channel, or speed or enhance deactivation. Activators are compounds that increase, open, activate, facilitate, enhance activation, sensitize or up regulate channel activity, or delay or slow inactivation. Such assays for inhibitors and activators also include, e.g., expressing recombinant KCNQ in cells or cell membranes (e.g., CHO cells expressing recombinant KCNQ channels) and then measuring flux of ions through the channel directly or indirectly. Alternatively, cells expressing endogenous KCNQ channels can be used in such assays (e.g., rat dorsal root ganglion cells expressing endogenous KCNQ channels).

To examine the extent of inhibition, samples or assays comprising a KCNQ channel are treated with a potential activator or inhibitor compound and are compared to control samples without the test compound. Control samples (untreated with test compounds) are assigned a relative KCNQ activity value of 100%. Inhibition of channels comprising a KCNQ subunit is achieved when the KCNQ activity value relative to the control is about 90%, preferably 50%, more preferably 0-25%. Activation of channels comprising a KCNQ subunit is achieved when the KCNQ activity value relative to the control is 110%, more preferably 150%, most preferably at least 200-500% higher or 1000% or higher.

An amount of compound that activates or inhibits a KCNQ channel, as described above, is a “potassium channel modulating amount” of the compound, which thereby reduces pain in a subject.

The term “modulate” refers to increases or decreases in the activity of a cell in response to exposure to a compound of the invention, e.g., the inhibition of proliferation and/or induction of differentiation of at least a sub-population of cells in an animal such that a desired end result is achieved, e.g., a therapeutic result. In preferred embodiments, this phrase is intended to include hyperactive conditions that result in pathological disorders.

The phrase “modulating ion flow,” or “increasing ion flow” in the context of assays for compounds affecting ion flux through a KCNQ channel, for the purposes of reducing pain in a subject, includes the determination of any parameter that is indirectly or directly under the influence of the channel. It includes physical, functional and chemical effects, e.g., changes in ion flux including radioisotopes, current amplitude, membrane potential, current flow, transcription, protein binding, phosphorylation, dephosphorylation, second messenger concentrations (cAMP, cGMP, Ca²⁺, IP₃), ligand binding, and other physiological effects such as hormone and neurotransmitter release, reduction in pain, as well as changes in voltage and current. The ion flux can be any ion that passes through a channel and analogues thereof, e.g., potassium, rubidium, sodium. Such functional, chemical or physical effects can be measured by any means known to those skilled in the art, e.g., patch clamping, voltage-sensitive dyes, whole cell currents, radioisotope efflux, inducible markers, in vivo pain assays such as formalin algesia or the hotplate test, and the like.

The term “interaction” or “interact” refers to an ionic, covalent, or a non-direct interaction.

“Biologically active” KCNQ refers to a KCNQ subunit that has the ability to form a potassium channel having the characteristic of voltage-gating tested as described above.

“Homomeric channel” refers to a KCNQ channel composed of identical alpha subunits, whereas “heteromeric channel” refers to a KCNQ channel composed of at least two different types of alpha subunit from a related gene family. Both homomeric and heteromeric channels can include auxiliary beta subunits. Typically, the channel is composed of four alpha subunits and the channel can be heteromeric or homomeric.

A “beta subunit” is a polypeptide monomer that is an auxiliary subunit of a potassium channel composed of alpha subunits; however, beta subunits alone cannot form a channel (see, e.g., U.S. Pat. No. 5,776,734). Beta subunits are known, for example, to increase the number of channels by helping the alpha subunits reach the cell surface, change activation kinetics, and change the sensitivity of natural ligands binding to the channels. Beta subunits can be outside of the pore region and associated with alpha subunits comprising the pore region. They can also contribute to the external mouth of the pore region.

The phrase “voltage-gated” activity or “voltage-gating” or “voltage dependence” refers to a characteristic of a potassium channel composed of individual polypeptide monomers or subunits. Generally, the probability of a voltage-gated potassium channel opening increases as a cell is depolarized. Voltage-gated potassium channels primarily allow efflux of potassium at membrane potentials more positive than the reversal potential for potassium (E_{K}) in typical cells, because they have greater probability of being open at such voltages. E_{K} is the membrane potential at which there is no net flow of potassium ions because the electrical potential (i.e., voltage potential) driving potassium efflux is balanced by the concentration gradient for potassium. The membrane potential of cells depends primarily on their potassium channels and is typically between −60 and −0.100 mV for mammalian cells. This value is also known as the “reversal potential” or the “Nernst” potential for potassium. Some voltage-gated potassium channels undergo inactivation, which can reduce potassium efflux at higher membrane potentials. Potassium channels can also allow potassium influx in certain instances when they remain open at membrane potentials negative to E_{K} (see, e.g., Adams & Nonner, in Potassium Channels, pp. 40-60 (Cook, ed., 1990)). The characteristic of voltage gating can be measured by a variety of techniques for measuring changes in current flow and ion flux through a channel, e.g., by changing the [K⁺] of the external solution and measuring the activation potential of the channel current (see, e.g., U.S. Pat. No. 5,670,335), by measuring current with patch clamp techniques or voltage clamp under different conditions, and by measuring ion flux with radiolabeled tracers or voltage-sensitive dyes under different conditions.

The phrase “functional effects” in the context of assays for testing compounds affecting a channel comprising KCNQ includes the determination of any parameter that is indirectly or directly under the influence of the channel. It includes physical and chemical effects, e.g., changes in ion flux and membrane potential, changes in ligand binding, and also includes other physiologic effects such as increases or decreases of transcription or hormone release.

“Determining the functional effect” refers to examining the effect of a compound that increases or decreases ion flux on a cell or cell membrane in terms of cell and cell membrane function. The ion flux can be any ion that passes through a channel and analogues thereof, e.g., potassium, rubidium. Preferably, the term refers to the functional effect of the compound on the channels, including those comprising KCNQ1, e.g., changes in ion flux including radioisotopes, current amplitude, membrane potential, current flow, transcription, protein binding, phosphorylation, dephosphorylation, second messenger concentrations (cAMP, cGMP, Ca²⁺, IP₃), ligand binding, and other physiological effects such as hormone and neurotransmitter release, as well as changes in voltage and current. Such functional effects can be measured by any means known to those skilled in the art, e.g., patch clamping, voltage-sensitive dyes, whole cell currents, radioisotope efflux, inducible markers, and the like.

The terms “isolated,” “purified,” or “biologically pure” refer to material that is substantially or essentially free from components that normally accompany it as found in its native state. Purity and homogeneity are typically determined using analytical chemistry techniques such as polyacrylamide gel electrophoresis or high performance liquid chromatography. A protein that is the predominant species present in a preparation is substantially purified. The term “purified” denotes that a nucleic acid or protein gives rise to essentially one band in an electrophoretic gel. Particularly, it means that the nucleic acid or protein is at least 85% pure, more preferably at least 95% pure, and most preferably at least 99% pure.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

A “peptide” is a sequence of at least two amino acids. Peptides can consist of short as well as long amino acid sequences, including proteins.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

The term “protein” refers to series of amino acid residues connected one to the other by peptide bonds between the alpha-amino and carboxy groups of adjacent residues.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I. The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The terms “identical” or percent “identity,” in the context of two or more nucleic acids or polypeptide sequences, refer to two or more sequences or subsequences that are the same or have a specified percentage of amino acid residues or nucleotides that are the same (i.e., 60% identity, preferably 65%, 70%, 75%, 85%, 90%, or 95% identity), when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length, or more preferably over a region that is 50-100 amino acids or nucleotides in length.

Algorithms suitable for determining percent sequence identity are the BLAST and BLAST 2.0 algorithms, which are described in Altschul et al, Nuc. Acids Res. 25:3389-3402 (1977) and Altschul et al., J. Mol. Biol. 215:403-410 (1990), respectively. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

A “label” is a composition detectable by spectroscopic, photochemical, biochemical, immunochemical, or chemical means. For example, useful labels include ³²P, fluorescent dyes, electron-dense reagents, enzymes (e.g., as commonly used in an ELISA), biotin, digoxigenin, or haptens and proteins for which antisera or monoclonal antibodies are available.

“Antibody” refers to a polypeptide comprising a framework region from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50 70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H1} by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990)).

An “anti-KCNQ1” antibody is an antibody or antibody fragment that specifically binds a polypeptide encoded by the KCNQ1 gene, cDNA, or a subsequence thereof.

A “chimeric antibody” is an antibody molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

The term “immunoassay” is an assay that uses an antibody to specifically bind an antigen. The immunoassay is characterized by the use of specific binding properties of a particular antibody to isolate, target, and/or quantify the antigen.

The phrase “specifically (or selectively) binds” to an antibody or “specifically (or selectively) immunoreactive with,” when referring to a protein or peptide, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Typically a specific or selective reaction will be at least twice background signal or noise and more typically more than 10 to 100 times background.

By “host cell” is meant a cell that contains an expression vector and supports the replication or expression of the expression vector. Host cells may be prokaryotic cells such as E. coli, or eukaryotic cells such as yeast, insect, amphibian, or mammalian cells such as CHO, HeLa and the like, e.g., cultured cells, explants, and cells in vivo.

The term “administration” or “administering” includes routes of introducing the compound(s) to a subject to perform their intended function. Examples of routes of administration which can be used include injection (subcutaneous, intravenous, parenterally, intraperitoneally, intrathecal), oral, inhalation, rectal and transdermal.

The term “effective amount” includes an amount effective, at dosages and for periods of time necessary, to achieve the desired result. An effective amount of compound may vary according to factors such as the disease state, age, and weight of the subject, and the ability of the compound to elicit a desired response in the subject. Dosage regimens may be adjusted to provide the optimum therapeutic response. An effective amount is also one in which any toxic or detrimental effects (e.g., side effects) of the angiogenesis inhibitor compound are outweighed by the therapeutically beneficial effects.

The phrases “systemic administration,” “administered systemically”, “peripheral administration” and “administered peripherally” as used herein mean the administration of a compound(s), drug or other material, such that it enters the patient's system and, thus, is subject to metabolism and other like processes, for example, subcutaneous administration.

The term “therapeutically effective amount” refers to that amount of the compound being administered sufficient to prevent development of or alleviate to some extent one or more of the symptoms of the condition or disorder being treated.

The terms “treating” and “treatment” refer to a method of alleviating or abating a disease and/or its attendant symptoms. In accordance with the present invention “treating” includes preventing, blocking, inhibiting, attenuating, protecting against, modulating, reversing the effects of and reducing the occurrence of e.g., the harmful effects of pain.

A therapeutically effective amount of compound (i.e., an effective dosage) may range from about 0.005 μg/kg to about 200 mg/kg, preferably about 0.1 mg/kg to about 200 mg/kg, more preferably about 10 mg/kg to about 100 mg/kg of body weight. The skilled artisan will appreciate that certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a compound can include a single treatment or, preferably, can include a series of treatments. In one example, a subject is treated with a compound in the range of between about 0.005 μg/kg to about 200 mg/kg of body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of a compound used for treatment may increase or decrease over the course of a particular treatment.

The term “chiral” refers to molecules which have the property of non-superimposability of the mirror image partner, while the term “achiral” refers to molecules which are superimposable on their mirror image partner.

The term “diastereomers” refers to stereoisomers with two or more centers of dissymmetry and whose molecules are not mirror images of one another.

The term “enantiomers” refers to two stereoisomers of a compound which are non-superimposable mirror images of one another. An equimolar mixture of two enantiomers is called a “racemic mixture” or a “racemate.”

The term “isomers” or “stereoisomers” refers to compounds which have identical chemical constitution, but differ with regard to the arrangement of the atoms or groups in space.

The term “prodrug” includes compounds with moieties which can be metabolized in vivo. Generally, the prodrugs are metabolized in vivo by esterases or by other mechanisms to active drugs. Examples of prodrugs and their uses are well known in the art (See, e.g., Berge et al. (1977) “Pharmaceutical Salts”, J. Pharm. Sci. 66:1-19). The prodrugs can be prepared in situ during the final isolation and purification of the compounds, or by separately reacting the purified compound in its free acid form or hydroxyl with a suitable esterifying agent. Hydroxyl groups can be converted into esters via treatment with a carboxylic acid. Examples of prodrug moieties include substituted and unsubstituted, branch or unbranched lower alkyl ester moieties, (e.g., propionic acid esters), lower alkenyl esters, di-lower alkyl-amino lower-alkyl esters (e.g., dimethylaminoethyl ester), acylamino lower alkyl esters (e.g., acetyloxymethyl ester), acyloxy lower alkyl esters (e.g., pivaloyloxymethyl ester), aryl esters (phenyl ester), aryl-lower alkyl esters (e.g., benzyl ester), substituted (e.g., with methyl, halo, or methoxy substituents) aryl and aryl-lower alkyl esters, amides, lower-alkyl amides, di-lower alkyl amides, and hydroxy amides. Preferred prodrug moieties are propionic acid esters and acyl esters. Prodrugs which are converted to active forms through other mechanisms in vivo are also included.

The term “subject” refers to animals such as mammals, including, but not limited to, primates (e.g., humans), cows, sheep, goats, horses, dogs, cats, rabbits, rats, mice and the like. In certain embodiments, the subject is a human.

Furthermore the indication of stereochemistry across a carbon-carbon double bond is also opposite from the general chemical field in that “Z” refers to what is often referred to as a “cis” (same side) conformation whereas “E” refers to what is often referred to as a “trans” (opposite side) conformation. With respect to the nomenclature of a chiral center, the terms “d” and “l” configuration are as defined by the IUPAC Recommendations. As to the use of the terms, diastereomer, racemate, epimer and enantiomer, these will be used in their normal context to describe the stereochemistry of preparations.

As used herein, the term “alkyl” refers to a straight-chained or branched hydrocarbon group containing 1 to 12 carbon atoms. The term “lower alkyl” refers to a C1-C6 alkyl chain. Examples of alkyl groups include methyl, ethyl, n-propyl, isopropyl, tert-butyl, and n-pentyl. Alkyl groups may be optionally substituted with one or more substituents.

The term “alkenyl” refers to an unsaturated hydrocarbon chain that may be a straight chain or branched chain, containing 2 to 12 carbon atoms and at least one carbon-carbon double bond. Alkenyl groups may be optionally substituted with one or more substituents.

The term “alkynyl” refers to an unsaturated hydrocarbon chain that may be a straight chain or branched chain, containing the 2 to 12 carbon atoms and at least one carbon-carbon triple bond. Alkynyl groups may be optionally substituted with one or more substituents.

The sp² or sp carbons of an alkenyl group and an alkynyl group, respectively, may optionally be the point of attachment of the alkenyl or alkynyl groups.

The term “alkoxy” refers to an —O-alkyl radical.

As used herein, the term “halogen” or “hal” means —F, —Cl, —Br or —I.

As used herein, the term “haloalkyl” means an alkyl group in which one or more (including all) of the hydrogen radicals are replaced by a halo group, wherein each halo group is independently selected from —F, —Cl, —Br, and —I. Representative haloalkyl groups include trifluoromethyl, bromomethyl, 1,2-dichloroethyl, 4-iodobutyl, 2-fluoropentyl, and the like.

The term “cycloalkyl” refers to a hydrocarbon 3-8 membered monocyclic or 7-14 membered bicyclic ring system having at least one saturated ring or having at least one non-aromatic ring, wherein the non-aromatic ring may have some degree of unsaturation. Cycloalkyl groups may be optionally substituted with one or more substituents. In one embodiment, 0, 1, 2, 3, or 4 atoms of each ring of a cycloalkyl group may be substituted by a substituent. Representative examples of cycloalkyl group include cyclopropyl, cyclopentyl, cyclohexyl, cyclobutyl, cycloheptyl, cyclooctyl, cyclononyl, cyclodecyl, cyclohexenyl, bicyclo[2.2.1]hept-2-enyl, dihydronaphthalenyl, benzocyclopentyl, cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl and the like.

The term “aryl” refers to a hydrocarbon monocyclic, bicyclic or tricyclic aromatic ring system. Aryl groups may be optionally substituted with one or more substituents. In one embodiment, 0, 1, 2, 3, 4, 5 or 6 atoms of each ring of an aryl group may be substituted by a substituent. Examples of aryl groups include phenyl, naphthyl, anthracenyl, fluorenyl, indenyl, azulenyl, and the like.

As used herein, the term “aralkyl” means an aryl group that is attached to another group by a (C₁-C₆)alkylene group. Aralkyl groups may be optionally substituted, either on the aryl portion of the aralkyl group or on the alkylene portion of the aralkyl group, with one or more substituents. Representative aralkyl groups include benzyl, 2-phenyl-ethyl, naphth-3-yl-methyl and the like.

As used herein, the term “alkylene” refers to an alkyl group that has two points of attachment. The term “(C₁-C₆)alkylene” refers to an alkylene group that has from one to six carbon atoms. Non-limiting examples of alkylene groups include methylene (—CH₂—), ethylene (—CH₂CH₂—), n-propylene (—CH₂CH₂CH₂—), isopropylene (—CH₂CH(CH₃)—), and the like.

The term “arylalkoxy” refers to an alkoxy substituted with aryl.

The term “heteroaryl” refers to an aromatic 5-8 membered monocyclic, 8-12 membered bicyclic, or 11-14 membered tricyclic ring system having 1-4 ring heteroatoms if monocyclic, 1-6 heteroatoms if bicyclic, or 1-9 heteroatoms if tricyclic, said heteroatoms selected from O, N, or S, and the remainder ring atoms being carbon (with appropriate hydrogen atoms unless otherwise indicated). Heteroaryl groups may be optionally substituted with one or more substituents. In one embodiment, 0, 1, 2, 3, or 4 atoms of each ring of a heteroaryl group may be substituted by a substituent. Examples of heteroaryl groups include pyridyl, 1-oxo-pyridyl, furanyl, benzo[1,3]dioxolyl, benzo[1,4]dioxinyl, thienyl, pyrrolyl, oxazolyl, oxadiazolyl, imidazolyl thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, and benzo[b]thienyl, 3H-thiazolo[2,3-c][1,2,4]thiadiazolyl, imidazo[1,2-d]-1,2,4-thiadiazolyl, imidazo[2,1-b]-1,3,4-thiadiazolyl, 1H,2H-furo[3,4-d]-1,2,3-thiadiazolyl, 1H-pyrazolo[5,1-c]-1,2,4-triazolyl, pyrrolo[3,4-d]-1,2,3-triazolyl, cyclopentatriazolyl, 3H-pyrrolo[3,4-c]isoxazolyl, 1H,3H-pyrrolo[1,2-c]oxazolyl, pyrrolo[2,1b]oxazolyl, and the like.

As used herein, the term “heteroaralkyl” means a heteroaryl group that is attached to another group by a (C₁-C₆)alkylene. Heteroaralkyl groups may be optionally substituted, either on the heteroaryl portion of the heteroaralkyl group or on the alkylene portion of the heteroaralkyl group, with one or more substituent. Representative heteroaralkyl groups include 2-(pyridin-4-yl)-propyl, 2-(thien-3-yl)-ethyl, imidazol-4-yl-methyl and the like.

The term “heterocycloalkyl” refers to a nonaromatic 3-8 membered monocyclic, 7-12 membered bicyclic, or 10-14 membered tricyclic ring system comprising 1-3 heteroatoms if monocyclic, 1-6 heteroatoms if bicyclic, or 1-9 heteroatoms if tricyclic, said heteroatoms selected from O, N, S, B, P or Si, wherein the nonaromatic ring system is completely saturated. Heterocycloalkyl groups may be optionally substituted with one or more substituents. In one embodiment, 0, 1, 2, 3, or 4 atoms of each ring of a heterocycloalkyl group may be substituted by a substituent. Representative heterocycloalkyl groups include piperidinyl, piperazinyl, 2-oxopiperazinyl, 2-oxopiperidinyl, 2-oxopyrrolidinyl, 4-piperidonyl, tetrahydropyranyl, tetrahydrothiopyranyl, tetrahydrothiopyranyl sulfone, morpholinyl, thiomorpholinyl, thiomorpholinyl sulfoxide, thiomorpholinyl sulfone, 1,3-dioxolane, tetrahydrofuranyl, tetrahydrothienyl, thiirenyl, thiadiazirinyl, dioxazolyl, 1,3-oxathiolyl, 1,3-dioxolyl, 1,3-dithiolyl, oxathiazinyl, dioxazinyl, dithiazinyl, oxadiazinyl, thiadiazinyl, oxazinyl, thiazinyl, 1,4-oxathiin, 1,4-dioxin, 1,4-dithiin, 1H-pyranyl, oxathiepinyl, 5H-1,4-dioxepinyl, 5H-1,4-dithiepinyl, 6H-isoxazolo[2,3-d]1,2,4-oxadiazolyl, 7aH-oxazolo[3,2-d]1,2,4-oxadiazolyl, and the like.

The term “alkylamino” refers to an amino substituent which is further substituted with one or two alkyl groups. The term “aminoalkyl” refers to an alkyl substituent which is further substituted with one or more amino groups. The term “hydroxyalkyl” or “hydroxylalkyl” refers to an alkyl substituent which is further substituted with one or more hydroxyl groups. The alkyl or aryl portion of alkylamino, aminoalkyl, mercaptoalkyl, hydroxyalkyl, mercaptoalkoxy, sulfonylalkyl, sulfonylaryl, alkylcarbonyl, and alkylcarbonylalkyl may be optionally substituted with one or more substituents.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Alkylating agents are any reagent that is capable of effecting the alkylation of the functional group at issue (e.g., oxygen atom of an alcohol, nitrogen atom of an amino group). Alkylating agents are known in the art, including in the references cited herein, and include allyl halides (e.g., methyl iodide, benzyl bromide or chloride), alkyl sulfates (e.g., methyl sulfate), or other alkyl group-leaving group combinations known in the art. Leaving groups are any stable species that can detach from a molecule during a reaction (e.g., elimination reaction, substitution reaction) and are known in the art, including in the references cited herein, and include halides (e.g., I—, Cl—, Br—, F—), hydroxy, alkoxy (e.g., —OMe, —O-t-Bu), acyloxy anions (e.g., —OAc, —OC(O)CF₃), sulfonates (e.g., mesyl, tosyl), acetamides (e.g., —NHC(O)Me), carbamates (e.g., N(Me)C(O)Ot-Bu), phosphonates (e.g., —OP(O)(OEt)₂), water or alcohols (protic conditions), and the like.

In certain embodiments, substituents on any group (such as, for example, alkyl, alkenyl, alkynyl, aryl, aralkyl, heteroaryl, heteroaralkyl, cycloalkyl, cyclyl, heterocycloalkyl, and heterocyclyl) can be at any atom of that group, wherein any group that can be substituted (such as, for example, alkyl, alkenyl, alkynyl, aryl, aralkyl, heteroaryl, heteroaralkyl, cycloalkyl, cyclyl, heterocycloalkyl, and heterocyclyl) can be optionally substituted with one or more substituents (which may be the same or different), each replacing a hydrogen atom. Examples of suitable substituents include, but are not limited to alkyl, alkenyl, cycloalkyl, heterocycloalkyl, aralkyl, heteroaralkyl, aryl, heteroaryl, halogen, haloalkyl, cyano, nitro, alkoxy, aryloxy, hydroxyl, hydroxylalkyl, oxo (i.e., carbonyl), carboxyl, formyl, alkylcarbonyl, alkylcarbonylalkyl, alkoxycarbonyl, alkylcarbonyloxy, aryloxycarbonyl, heteroaryloxy, heteroaryloxycarbonyl, thio, mercapto, mercaptoalkyl, arylsulfonyl, amino, aminoalkyl, dialkylamino, alkylcarbonylamino, alkylaminocarbonyl, alkoxycarbonylamino, alkylamino, arylamino, diarylamino, alkylcarbonyl, or arylamino-substituted aryl; arylalkylamino, aralkylaminocarbonyl, amido, alkylaminosulfonyl, arylaminosulfonyl, dialkylaminosulfonyl, alkylsulfonylamino, arylsulfonylamino, imino, carbamido, carbamyl, thioureido, thiocyanato, sulfoamido, sulfonylalkyl, sulfonylaryl, or mercaptoalkoxy.

Additional suitable substituents are alkyl, alkenyl, alkynyl, aryl, aralkyl, heteroaryl, heteroaralkyl, cycloalkyl, cyclyl, heterocycloalkyl, and heterocyclyl and include, without limitation halogen, CN, NO₂, OR¹⁵, SR¹⁵, S(O)₂OR¹⁵, NR¹⁵R¹⁶, C₁-C₂ perfluoroalkyl, C₁-C2 perfluoroalkoxy, 1,2-methylenedioxy, (═O), (═S), (═NR¹⁵), C(O)OR¹⁵, C(O)NR¹⁵R¹⁶, OC(O)NR¹⁵R¹⁶, NR¹⁵C(O)NR¹⁵R¹⁶, C(NR¹⁶)NR¹⁵R¹⁶, NR¹⁵C(NR¹⁶)NR¹⁵R¹⁶, S(O)₂NR¹⁵R¹⁶, R¹⁷, C(O)H, C(O)R¹⁷, NR¹⁵C(O)R¹⁷, Si(R¹⁵)₃, OSi(R¹⁵)₃, Si(OH)₂R¹⁵, B(OH)₂, P(O)(OR¹⁵)₂, S(O)R¹⁷, or S(O)₂R¹⁷. Each R¹⁵ is independently hydrogen, C₁-C₆ alkyl optionally substituted with cycloalkyl, aryl, heterocyclyl, or heteroaryl. Each R¹⁶ is independently hydrogen, C₃-C₆ cycloalkyl, aryl, heterocyclyl, heteroaryl, C₁-C₄ alkyl or C₁-C₄ alkyl substituted with C₃-C₆cycloalkyl, aryl, heterocyclyl or heteroaryl. Each R¹⁷ is independently C₃-C₆ cycloalkyl, aryl, heterocyclyl, heteroaryl, C₁-C₄ alkyl or C₁-C₄ alkyl substituted with C₃-C₆ cycloalkyl, aryl, heterocyclyl or heteroaryl. Each C₃-C₆ cycloalkyl, aryl, heterocyclyl, heteroaryl and C₁-C₄ alkyl in each R¹⁵, R¹⁶ and R¹⁷ can optionally be substituted with halogen, CN, C₁-C₄ alkyl, OH, C₁-C₄ alkoxy, COOH, C(O)OC₁-C₄ alkyl, NH₂, C₁-C₄ alkylamino, or C₁-C₄ dialkylamino.

### Methods of Treatment

In one aspect, the invention provides a method of treating a KCNQ related disorder in a subject, comprising administering to said subject, an effective amount of a KCNQ polypeptide channel activity modulator, such that said subject is treated for said disorder

In one aspect, the invention provides a method of treating a KCNQ related disorder in a subject, comprising administering to said subject, an effective amount of a KCNQ polypeptide channel activity modulator, such that said subject is treated for said disorder; wherein the modulator of a KCNQ polypeptide channel is an organozinc compound.

In another aspect, the invention provides a method of treating a KCNQ related disorder in a subject, wherein the subject has been identified as in need of treatment for a KCNQ related disorder, comprising administering to said subject in need thereof, an effective amount of a KCNQ polypeptide channel activity modulator, such that said subject is treated for said disorder, wherein the modulator of a KCNQ polypeptide channel is an organozinc compound.

In one embodiment, the invention provides a method wherein the KCNQ polypeptide channel activity modulator interacts with a KCNQ polypeptide channel.

In certain instances, the KCNQ polypeptide channel is a potassium channel.

In certain instances, the organozinc compound is a zinc ionophore.

In certain embodiments, the organozinc compound is selected from zinc pyrithione (Zn-Py), zinc heterocyclic amines, zinc dithiocarbamates, and zinc vitamins.

In a further embodiment, the organozinc compound is Zinc Pyrithione (ZnPy):

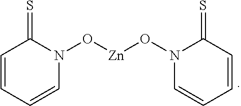

In another further embodiment, the zinc-heterocyclic amine is selected from zinc-5,7-Diiodo-8-hydroxyquinoline and zinc-8-Hydroxyquinoline.

In still another further embodiment, the zinc-dithiocarbamates are selected from zinc-pyrrolidine dithiocarbamate, zinc-diethyldithiocarbamate, zinc-disulfiram and zinc-dimethyldithiocarbamate.

In another further embodiment, the zinc-vitamin is selected from zinc-vitamin E and zinc-vitamin A.

In one embodiment, the organozinc compound is a zinc moiety bound, coordinated, or chelated to a compound of formula I:

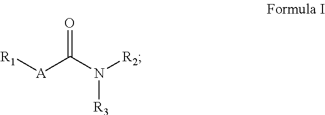

wherein:

A is a bond, CH₂, CHR_{b}, CH₂S, CHR_{b}S, CH₂O, CH₂NR_{c}, or NH;
R_{b} is alkyl;
R_{c} is H or S(O)ₘ-aryl;

R₁ is an optionally substituted alkyl, an optionally substituted aryl, an optionally substituted cycloalkyl, or an optionally substituted heteroaryl;

R₂ is an optionally substituted alkyl, an optionally substituted alkenyl, an optionally substituted aryl, an optionally substituted cycloalkyl, an optionally substituted heteroaryl, an optionally substituted heterocyclic; an optionally substituted aralkyl,

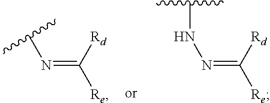

R₃ is H or alkyl;
each R_{d} and Rₑ is independently an optionally substituted alkyl, an optionally substituted aryl, or R_{d} and Rₑ together form an optionally substituted cycloalkyl; and
m is 0, 1, or 2.

In another embodiment, the organozinc compound is zinc moiety bound, coordinated, or chelated to a compound of formula II:

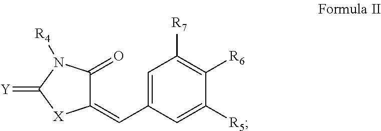

wherein:

R₄ is H, an optionally substituted alkyl, an optionally substituted alkenyl, alkynyl, allyl, or an optionally substituted aryl;

R₅ is H, hal, or hydroxyl;

R₆ is H, hal, hydroxyl, NH₂, a mono- or di-substituted amine, or an optionally substituted alkoxy;

R₇ is H, hal, hydroxyl, an optionally substituted alkoxy, or nitro;

X is S or NRₐ;

Y is O, S, or NRₐ; and
each Rₐ is independently H or an optionally substituted aryl.

In certain embodiments, the organozinc compound is a zinc moiety bound, coordinated, or chelated to the following: N-Benzo[g]quinolin-4-yl-N′-(2-diethylamino-ethyl)-benzene-1,4-diamine; 2-[2-(3,4-Dihydroxy-phenyl)-2-oxo-ethylsulfanyl]-4,6-dimethyl-nicotinonitrile; 2-[2-(4-Methoxy-phenyl)-2-oxo-ethylsulfanyl]-4-(5-methyl-furan-2-yl)-5,6,7,8-tetrahydro-quinoline-3-carbonitrile; 6-Methyl-4-(5-methyl-furan-2-yl)-2-(2-oxo-2-phenyl-ethylsulfanyl)-nicotinonitrile; 2-(2-Oxo-2-thiophen-2-yl-ethylsulfanyl)-4-pyridin-4-yl-5,6,7,8-tetrahydro-quinoline-3-carbonitrile; 2-(3,5-Diiodo-2-methoxy-phenyl)-2,3,5,6,7,8-hexahydro-1H-benzn[4,5]thieno[2,3-d]pyrimidin-4-one; 2,2,2-Trifluoro-1-[1-(2,2,2-trifluoro-1-hydroxy-1-trifluoromethyl-ethyl)-1H-pyrrol-2-yl]-ethanone; 1,5-Diphenyl-1H-pyrazole-3-carboxylic acid tert-butylamide; 3-(4-Bromo-phenyl)-5-(3-phenyl-allylidene)-dihydro-pyrimidine-2,4-dione; 2-Amino-7-hydroxy-6-[(2-iodo-phenylimino)-methyl]-4-phenyl-4H-chromene-3-carbonitrile; 3-(1H-Benzoimidazol-2-yl)-6-nitro-chromen-2-ylideneamine; 6-Methoxy-3-(4-nitro-phenyl)-chromen-2-one; 2-(Benzo[1,2,5]thiadiazol-4-yliminomethyl)-benzo[b]thiophen-3-ol; 4-[3-(4-Bromo-phenyl)-3-oxo-propenylamino]-N-(4,6-dimethyl-pyrimidin-2-yl)-benzenesulfonamide; 2-[(5-Nitro-furan-2-ylmethylene)-amino]-benzamide; 2-Benzo[4,5]imidazo[1,2-c]quinazolin-6-yl-phenylamine; 1-(3,5-Dimethyl-phenyl)-5-(3-phenyl-allylidene)-pyrimidine-2,4,6-trione; 4-(4-Cyclohexyl-phenyl)-thiazol-2-ylamine; or 4-(4-Cyclohexyl-phenyl)-thiazol-2-ylamine.

In one embodiment, the KCNQ related disorder is pain, epilepsy, myokymia or cardiac disease. In a preferred embodiment, the disorder is pain. In certain instances, the pain is somatic pain. In other instances, the pain is cutaneous. In still other instances, the pain is visceral.

In certain embodiments, the pain is caused by a burn, a bruise, an abrasion, a laceration, a broken bone, a torn ligament, a torn tendon, a torn muscle, a viral infection, a bacterial infection, a protozoal infection, a fungal infection, contact dermatitis, inflammation, or cancer. In a further embodiment, the inflammation is caused by trauma, infection, surgery, burns, or diseases with an inflammatory component.

In another embodiment, the pain is neuropathic. In a further embodiment, the neuropathic pain is caused by injury to the central or peripheral nervous system due to cancer, HIV infection, tissue trauma, infection, autoimmune disease, diabetes, arthritis, diabetic neuropathy, trigeminal neuralgia or drug administration.

In other embodiments, the cardiac disease is long QT syndrome (LQTS), heart failure, fibrillation, or arrhythmia.

In one embodiment, the KCNQ channel is a heteromeric channel. In another embodiment, the KCNQ channel is a homomeric channel.

In certain embodiments, the KCNQ channel comprises at least one of KCNQ 1, KCNQ 2, KCNQ 3, KCNQ 4, and KCNQ 5; preferably KCNQ 1.

In one feature of the invention, the effective amount of organozinc compound ranges from about 0.005 μg/kg to about 200 mg/kg. In one instance, the effective amount of organozinc compound ranges from about 0.1 mg/kg to about 200 mg/kg. In a further instance, the effective amount of organozinc compound ranges from about 10 mg/kg to 100 mg/kg.

In another feature, the organozinc compound is administered intravenously, intramuscularly, subcutaneously, intracerebroventricularly, orally or topically.

In one embodiment, the interaction of the organozinc compound with a KCNQ channel causes a hyperpolarization shift of voltage sensitivity of the KCNQ channel. In another embodiment, the interaction of the organozinc compound with a KCNQ channel reduces the deactivation of the KCNQ channel. In still another embodiment, the interaction of the organozinc compound with a KCNQ channel causes a conformational change of the KCNQ channel. In a further embodiment, the conformational change is an opening of the KCNQ channel.

In certain embodiments, the modulation of the activity is inhibited or stimulated.

In another embodiment, the organozinc compound and the KCNQ channel interact to provide an increase or a decrease in voltage potential.

In one embodiment, the KCNQ channel comprises a S5 protein, a S6 protein, and a pore region. In a further embodiment, the organozinc compound interacts with the S5-pore-S6 protein region of the KCNQ channel.

In certain embodiments, the interaction of the KCNQ channel with the organozinc compound results in opening of the pore and a change in voltage.

In one embodiment, the organozinc compound interacts with cysteine residues. In another embodiment, the organozinc compound interacts with alanine residues. In certain instances, the compound interacts with Trp and Gly residues.

In certain embodiments, the interaction between the organozinc compound and the KCNQ channel takes place in a cell. In one embodiment, the cell is in a mammal. In another embodiment, the cell is from a mammal. In a further embodiment, the mammal is a human or a rodent. In certain instances, the cell is in vitro.

In another embodiment, the subject is a mammal; preferably a primate or human.

In certain embodiments, the organozinc compound is administered alone or in combination with one or more other pain therapeutics.

In another aspect, the invention provides a method of protecting against the effects of pain comprising, administering to a subject in need thereof, a pharmaceutically effective amount of a KCNQ polypeptide channel activity modulator; wherein the modulator is an organozinc compound. In one embodiment, the invention provides a method wherein the KCNQ polypeptide channel activity modulator interacts with a KCNQ polypeptide channel.

In one aspect, the invention provides a method of modulating the activity of a potassium channel in a subject, comprising contacting a potassium channel with an organozinc compound. In another aspect, the invention provides a method of modulating the activity of a potassium channel in a subject, comprising administering to said subject an effective amount of an organozinc compound.

In yet another aspect, the invention provides a method for identifying a compound which modulates the activity of a KCNQ polypeptide channel, the method comprising: a) contacting a KCNQ polypeptide channel with a compound under conditions suitable for modulation of the activity of the KCNQ polypeptide channel; and b) detecting modulation of the activity of the KCNQ polypeptide channel by the compound; wherein the compound chemically interacts with the KCNQ polypeptide channel.

In one embodiment, the KCNQ polypeptide channel is KCNQ 1. In another embodiment, the interaction of the compound with the KCNQ polypeptide channel is a binding interaction. In a further embodiment, the binding interaction is ionic, covalent, or a non-direct interaction.

In a further embodiment, the invention provides a method further comprising the step of testing the compound for biological activity.

In one embodiment, the compound is identified using a rubidium efflux assay. In a further embodiment, the rubidium efflux assay is non-radioactive.

In another aspect, the invention provides a method of treating a KCNQ mutation disorder in a subject, comprising administering to the subject, an effective amount of a KCNQ polypeptide channel activity potentiator, such that said subject is treated for said disorder, wherein the potentiator of a KCNQ polypeptide channel is an organozinc compound.

In another aspect, the invention provides a method of treating a KCNQ mutation disorder in a subject, wherein the subject has been identified as in need of treatment for a KCNQ mutation disorder, comprising administering to said subject in need thereof, an effective amount of a KCNQ polypeptide channel activity potentiator, such that said subject is treated for said disorder; wherein the potentiator of a KCNQ polypeptide channel is an organozinc compound.

In certain embodiments, the invention provides a method wherein the KCNQ polypeptide channel activity potentiator interacts with a KCNQ polypeptide channel.

The instant invention demonstrates for the first time that the organozinc compounds described above, which are modulators of KCNQ polypeptide channels, alleviate pain. The present invention provides a mechanism for treating pain disorders, and an assay for identifying compounds that open KCNQ polypeptide channels and reduce pain. Modulation of KCNQ-type channels by organozinc compounds therefore represents a novel approach to the treatment of pain, including both somatic and neuropathic pain.

In accordance with the present invention the organozinc compounds protect against neuronal cell loss in stroke patients. For example, zinc pyrithione demonstrates neuroprotective properties, showing protection against cell loss in the selectively vulnerable zone of the CA1 region of the hippocampus in a rat model of severe global ischemia. In the mouse model of severe focal ischemia, zinc pyrithione demonstrates neuroprotective properties, significantly decreasing brain infarct volume and neurological deficit.

The organozinc compounds of the present invention are versatile in their efficacy and can be used to treat diseases associated with the human eye including hereditary degenerative retinopathies, including macular degeneration and retinitis pigmentosa, for example. Other diseases of the eye treatable with the zinc ionophores of the present invention include, but are not limited to cataracts (diabetic and chemically induced, for example), glaucoma, inflammatory eye diseases, corneal apoptosis associated with transplantation and Fuchs' dystrophy.

The organozinc compounds of the present invention can also be used to treat neurodegenerative diseases such as Alzheimer's Disease, Parkinson's Disease, Huntington's Disease, Amyotrophic lateral sclerosis (ALS) and multiple sclerosis, for example.

The organozinc compounds of the present invention also exhibit anti-epileptic efficacy. In accordance with the present invention “anti-epileptic” means “anti-convulsive” and “anti-seizure”. For example, zinc diethyldithiocarbamate demonstrates an anti-seizure effect in a mouse model of seizure. Using the total elimination of tonic extension as the principal criterion for anti-seizure efficacy, zinc diethyldithiocarbamate showed statistically significant ability to block tonic seizures. Accordingly, the present invention provides a method for attenuating both the duration and severity of seizures in mammalian subjects, including humans.

In certain embodiments, the compounds of the invention are used to treat pain, epilepsy, myokymia or cardiac disease. In a preferred embodiment, the disorder is pain. In certain instances, the pain is somatic pain. In other instances, the pain is cutaneous. In still other instances, the pain is visceral.

In certain embodiments, the pain is caused by a burn, a bruise, an abrasion, a laceration, a broken bone, a torn ligament, a torn tendon, a torn muscle, a viral infection, a bacterial infection, a protozoal infection, a fungal infection, contact dermatitis, inflammation, or cancer. In a further embodiment, the inflammation is caused by trauma, infection, surgery, burns, or diseases with an inflammatory component.

In another embodiment, the pain is neuropathic. In a further embodiment, the neuropathic pain is caused by injury to the central or peripheral nervous system due to cancer, HIV infection, tissue trauma, infection, autoimmune disease, diabetes, arthritis, diabetic neuropathy, trigeminal neuralgia or drug administration.

In another embodiment, the organozinc compounds can be used to treat or prevent acute or chronic pain. Examples of pain treatable or preventable using the such compounds include, but are not limited to, cancer pain, central pain, labor pain, myocardial infarction pain, pancreatic pain, colic pain, post-operative pain, headache pain, muscle pain, arthritic pain, and pain associated with a periodontal disease, including gingivitis and periodontitis.

The pain to be treated or prevented may be associated with inflammation associated with an inflammatory disease, which can arise where there is an inflammation of the body tissue, and which can be a local inflammatory response and/or a systemic inflammation. For example, the organozinc compounds can be used to treat, or prevent pain associated with inflammatory disease including, but not limited to: organ transplant rejection; reoxygenation injury resulting from organ transplantation (see Grupp et al., J. Mol, Cell Cardiol. 31:297 303 (1999)) including, but not limited to, transplantation of the heart, lung, liver, or kidney; chronic inflammatory diseases of the joints, including arthritis, rheumatoid arthritis, osteoarthritis and bone diseases associated with increased bone resorption; inflammatory bowel diseases, such as ileitis, ulcerative colitis, Barrett's syndrome, and Crohn's disease; inflammatory lung diseases, such as asthma, adult respiratory distress syndrome, and chronic obstructive airway disease; inflammatory diseases of the eye, including corneal dystrophy, trachoma, onchocerciasis, uveitis, sympathetic ophthalmitis and endophthalmitis; chronic inflammatory disease of the gum, including gingivitis and periodontitis; tuberculosis; leprosy; inflammatory diseases of the kidney, including uremic complications, glomerulonephritis and nephrosis; inflammatory disease of the skin, including sclerodermatitis, psoriasis and eczema; inflammatory diseases of the central nervous system, including chronic demyelinating diseases of the nervous system, multiple sclerosis, AIDS-related neurodegeneration and Alzheimer's disease, infectious meningitis, encephalomyelitis, Parkinson's disease, Huntington's disease, amyotrophic lateral sclerosis and viral or autoimmune encephalitis; autoimmune diseases, including Type I and Type II diabetes mellitus; diabetic complications, including, but not limited to, diabetic cataract, glaucoma, retinopathy, nephropathy (such as microaluminuria and progressive diabetic nephropathy), polyneuropathy, mononeuropathies, autonomic neuropathy, gangrene of the feet, atherosclerotic coronary arterial disease, peripheral arterial disease, nonketotic hyperglycemic-hyperosmolar coma, foot ulcers, joint problems, and a skin or mucous membrane complication (such as an infection, a shin spot, a candidal infection or necrobiosis lipoidica diabeticorum); immune-complex vasculitis, and systemic lupus erythematosus (SLE); inflammatory disease of the heart, such as cardiomyopathy, ischemic heart disease hypercholesterolemia, and arteriosclerosis; as well as various other diseases that can have significant inflammatory components, including preeclampsia, chronic liver failure, brain and spinal cord trauma, and cancer. The organozinc compounds can also be used for inhibiting, treating, or preventing pain associated with inflammatory disease that can, for example, be a systemic inflammation of the body, exemplified by gram-positive or gram negative shock, hemorrhagic or anaphylactic shock, or shock induced by cancer chemotherapy in response to pro-inflammatory cytokines, e.g., shock associated with pro-inflammatory cytokines. Such shock can be induced, e.g., by a chemotherapeutic agent that is administered as a treatment for cancer.

In certain instances, the compounds utilized in the invention to treat KCNQ related disorder disorders, such as pain, include the compounds of Table 1. In certain instances, such compounds have an ionic bond with zinc, or are coordinated or chelated to zinc to form an organozinc compound. The structures depicted herein, including the Table 1 structures, may contain certain —NH—, —NH₂ (amino) and —OH (hydroxyl) groups where the corresponding hydrogen atom(s) do not explicitly appear, however they are to be read as —NH—, —NH₂ or —OH as the case may be. In certain instances, nitrogen, oxygen, or sulfur may be bound, coordinated, or chelated to zinc. The structures depicted herein, including the Table 1 structures, may contain a methyl group depicted as -Me, —CH₃, or an organic bond without further substituents.

TABLE 1

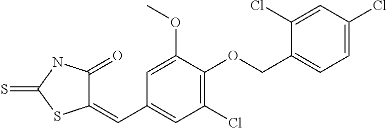

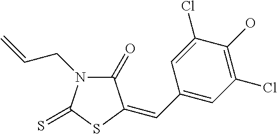

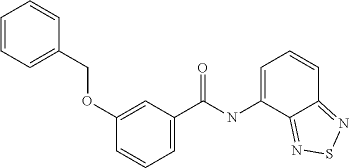

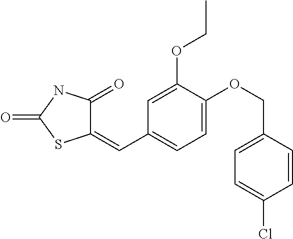

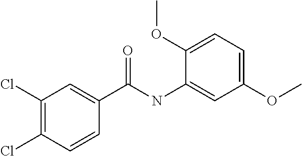

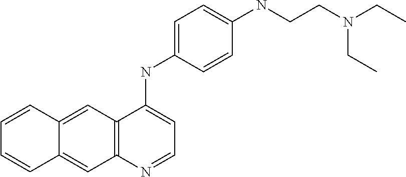

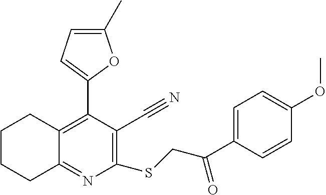

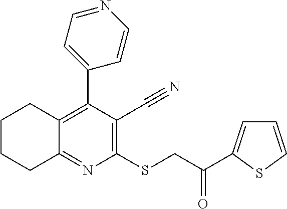

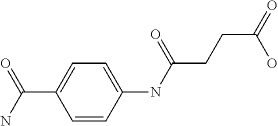

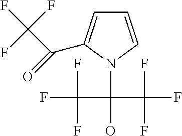

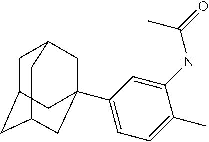

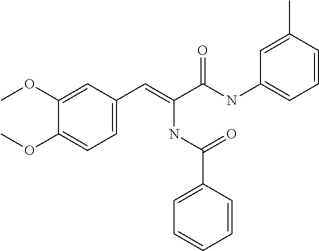

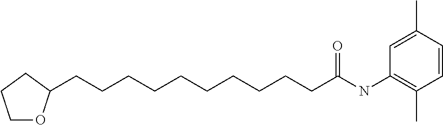

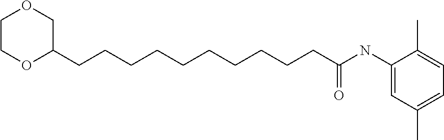

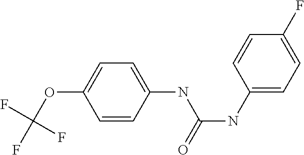

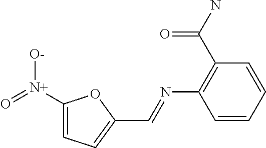

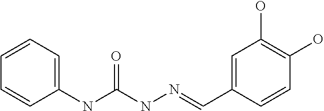

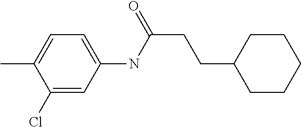

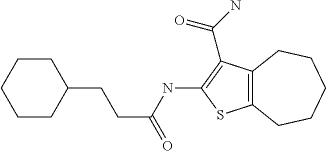

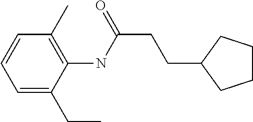

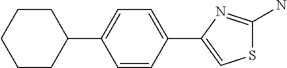

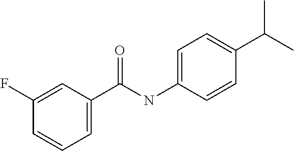

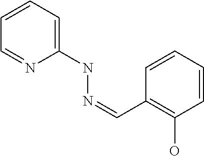

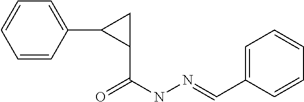

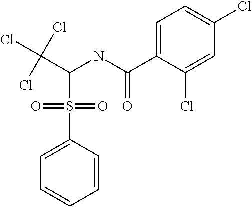

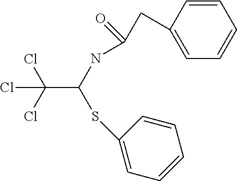

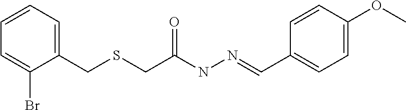

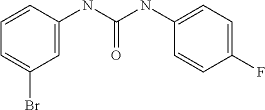

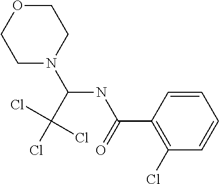

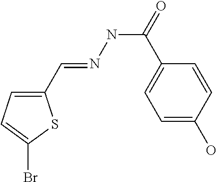

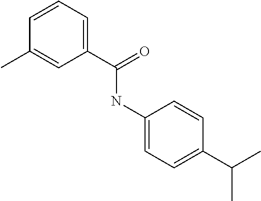

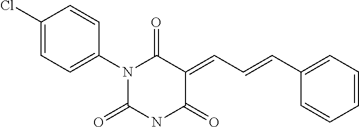

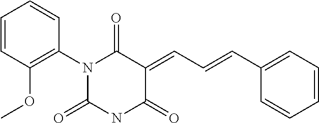

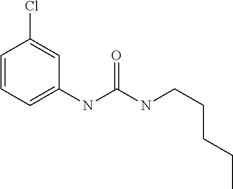

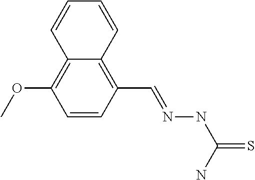

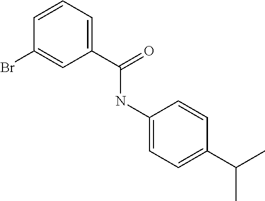

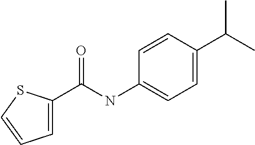

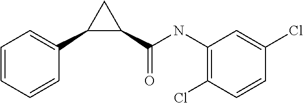

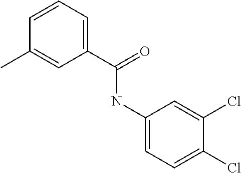

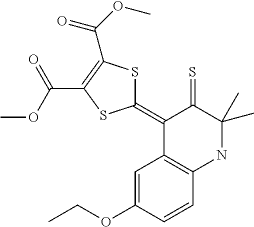

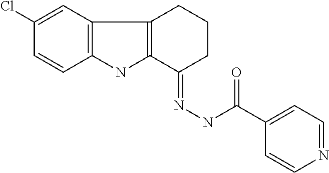

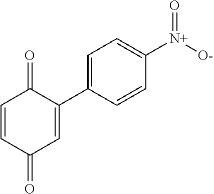

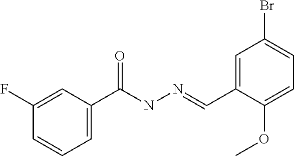

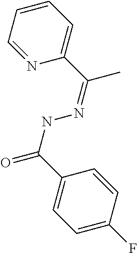

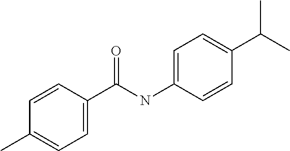

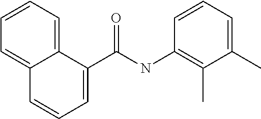

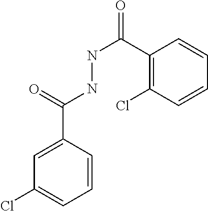

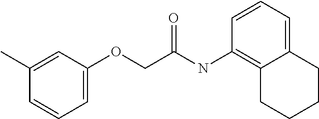

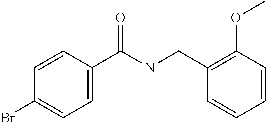

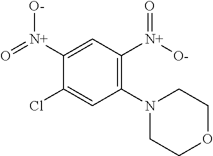

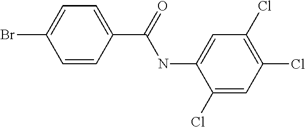

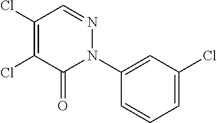

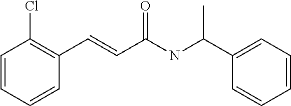

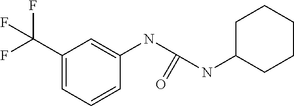

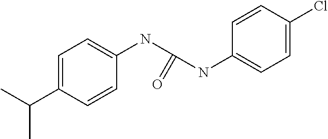

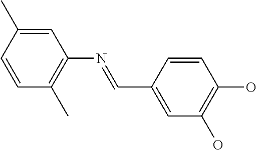

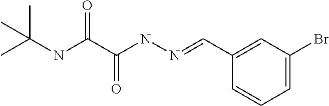

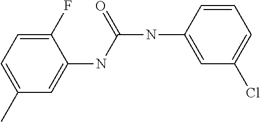

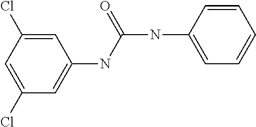

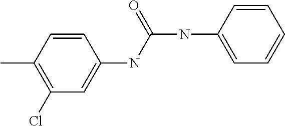

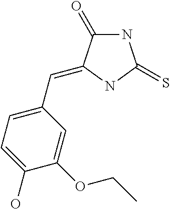

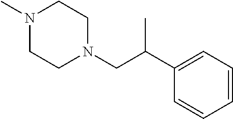

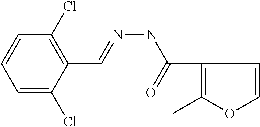

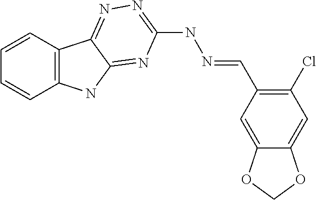

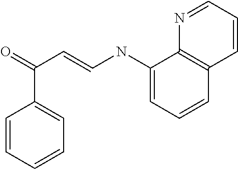

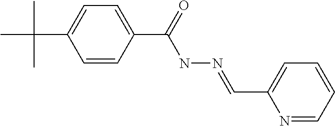

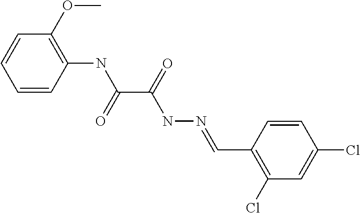

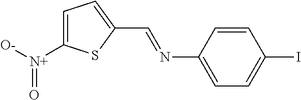

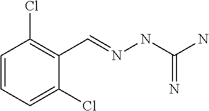

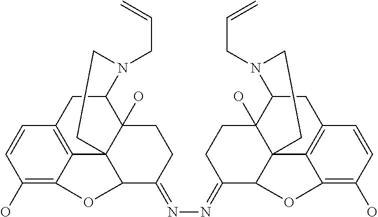

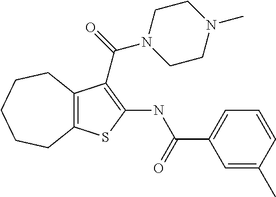

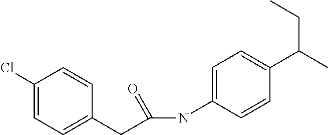

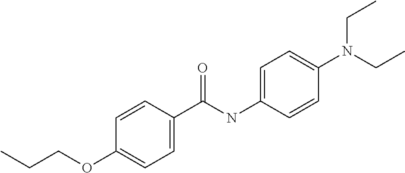

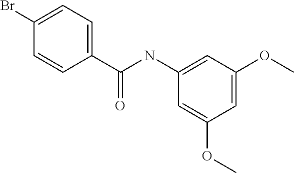

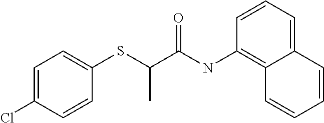

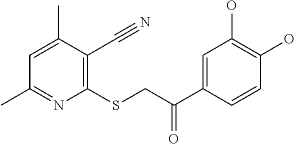

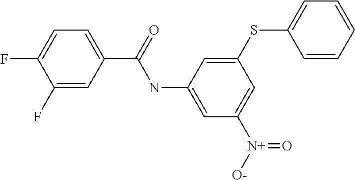

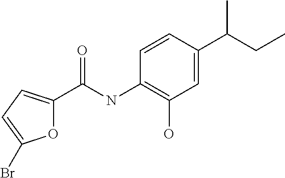

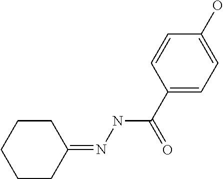

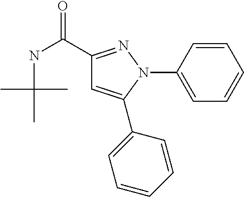

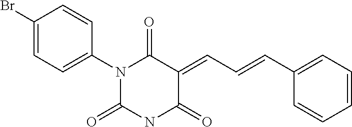

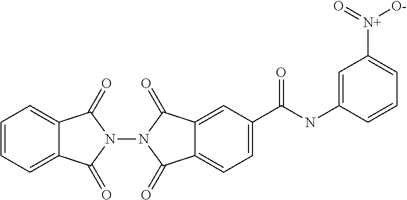

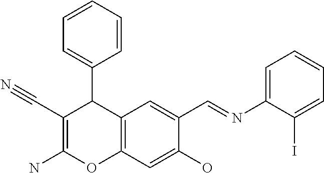

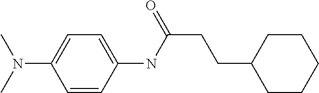

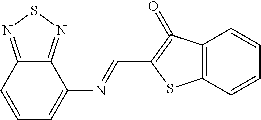

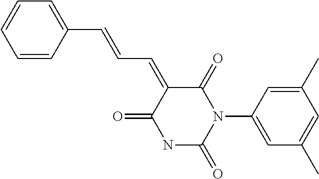

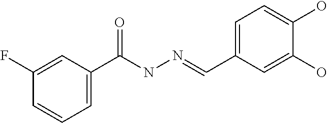

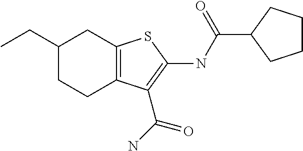

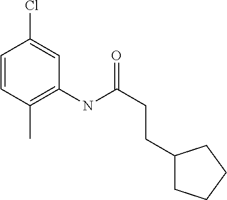

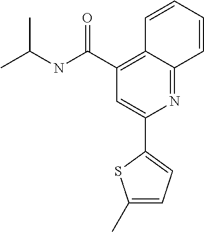

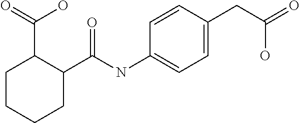

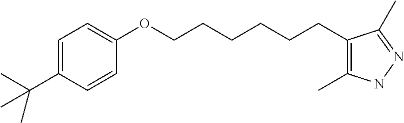

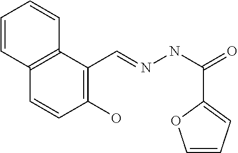

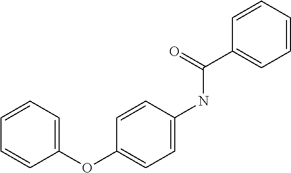

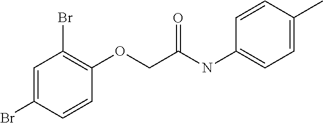

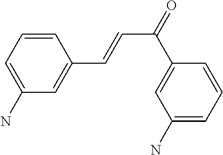

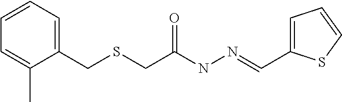

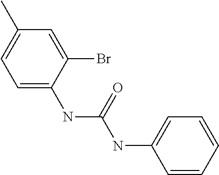

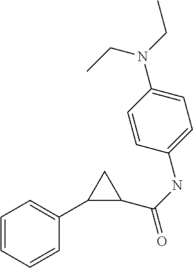

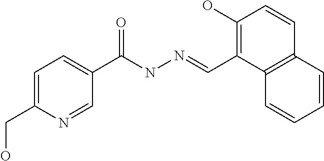

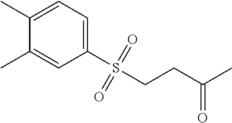

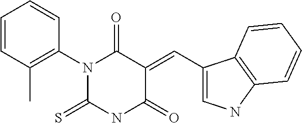

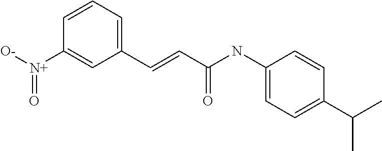

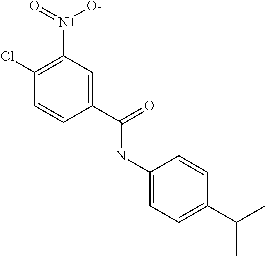

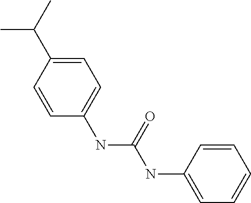

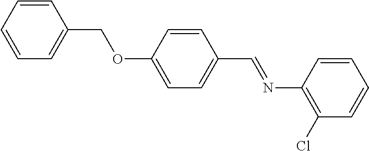

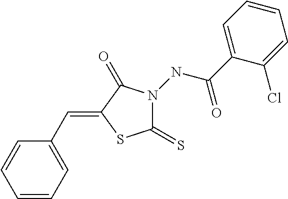

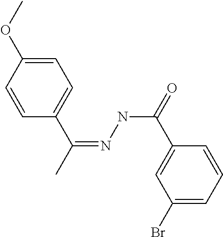

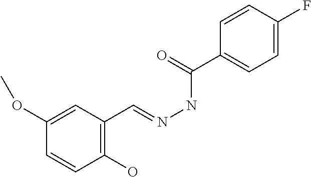

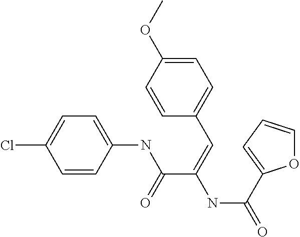

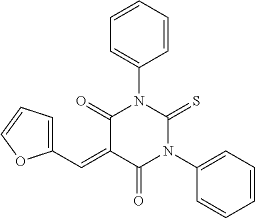

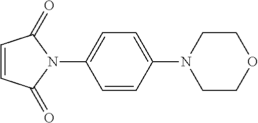

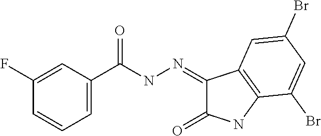

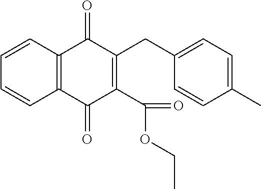

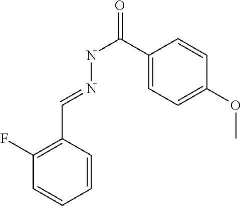

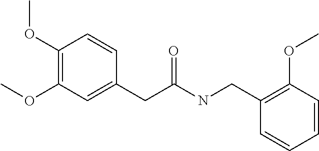

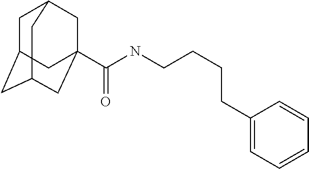

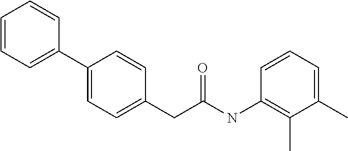

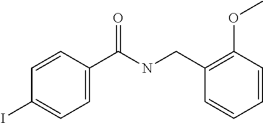

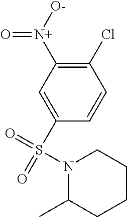

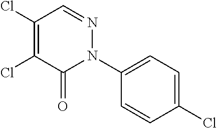

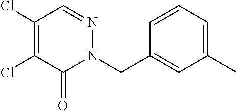

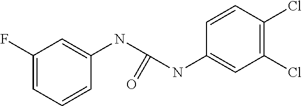

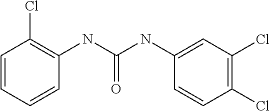

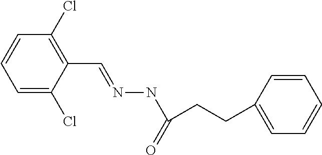

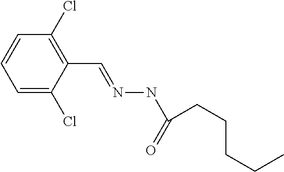

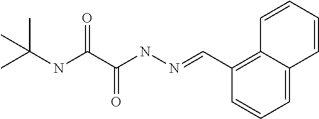

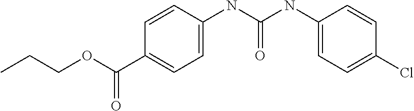

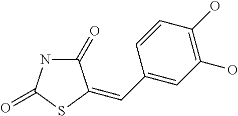

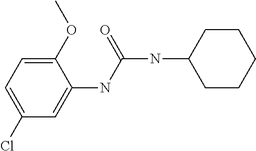

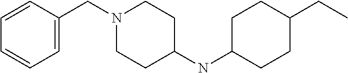

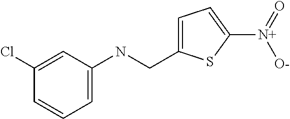

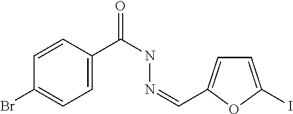

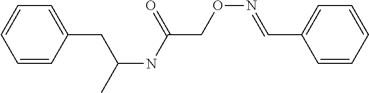

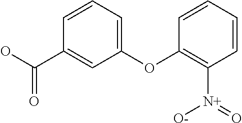

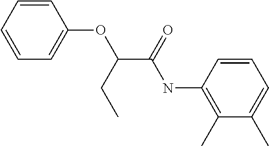

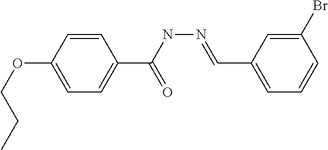

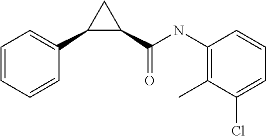

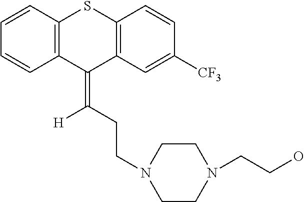

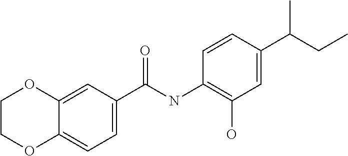

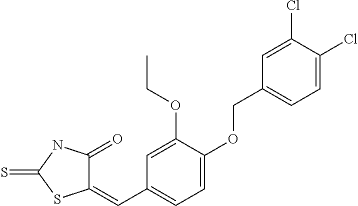

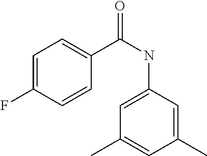

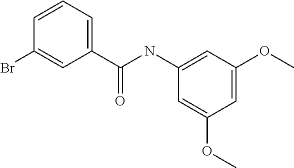

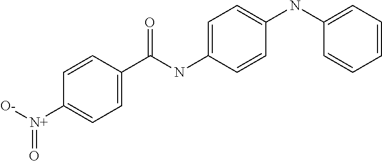

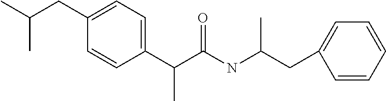

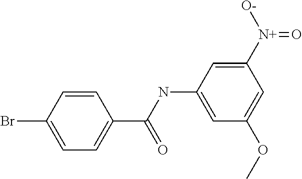

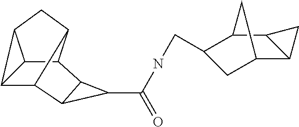

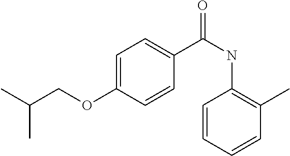

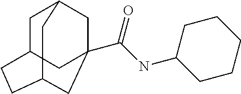

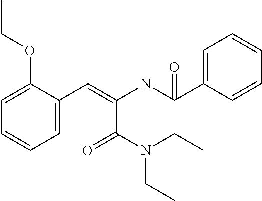

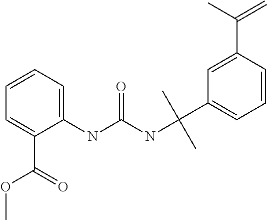

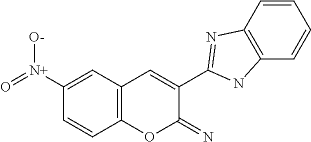

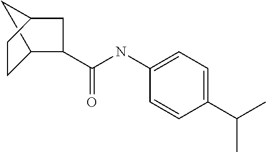

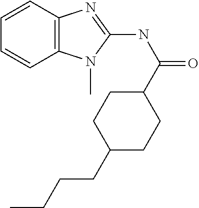

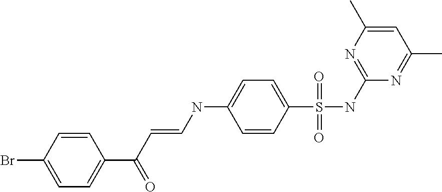

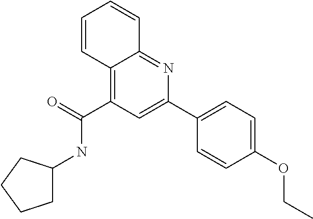

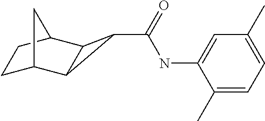

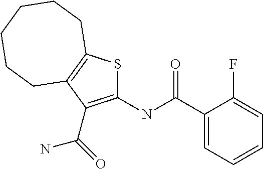

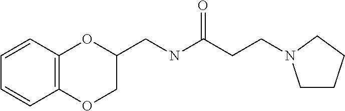

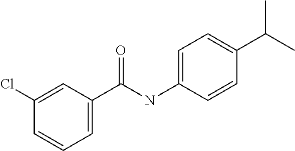

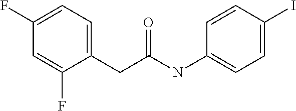

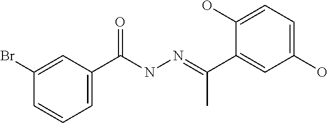

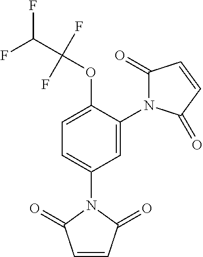

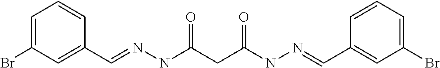

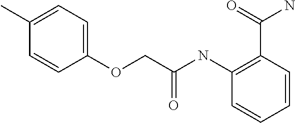

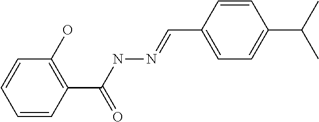

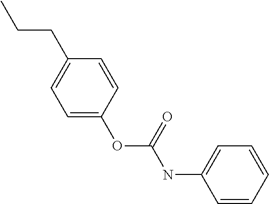

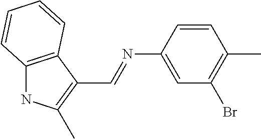

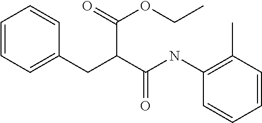

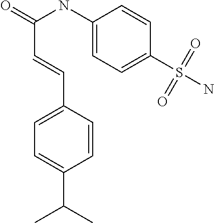

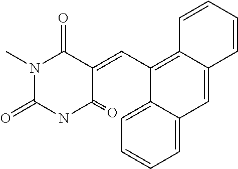

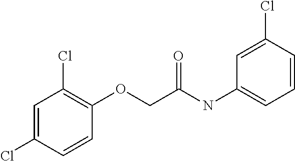

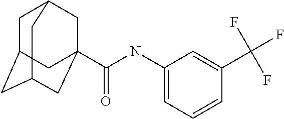

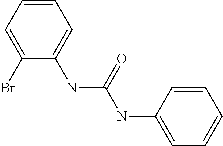

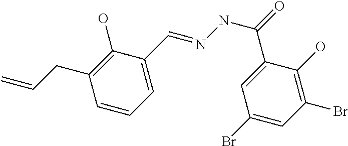

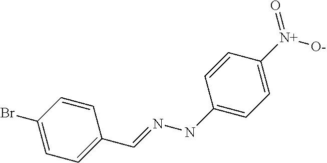

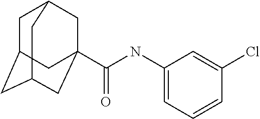

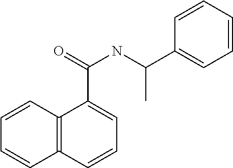

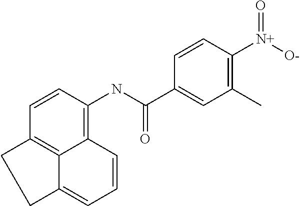

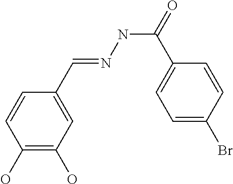

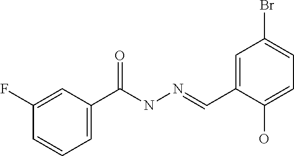

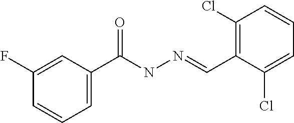

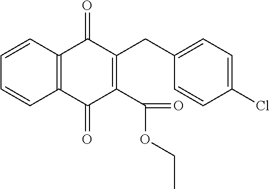

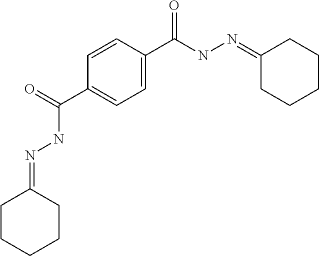

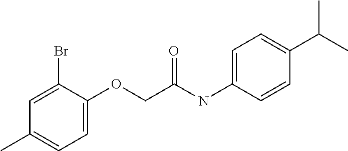

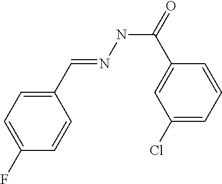

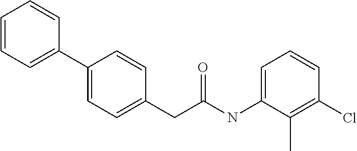

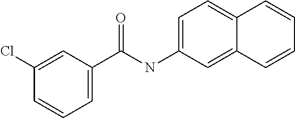

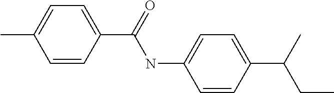

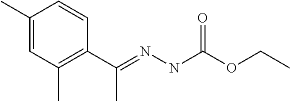

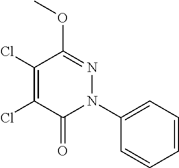

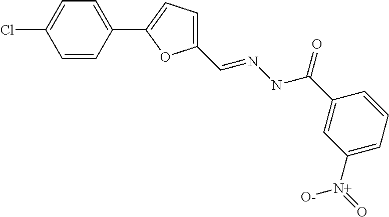

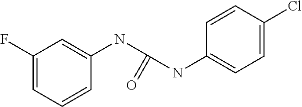

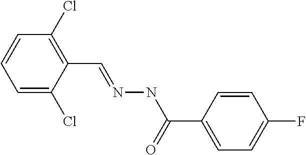

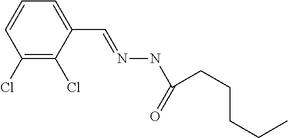

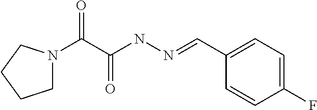

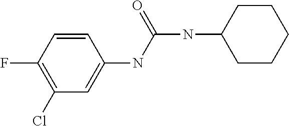

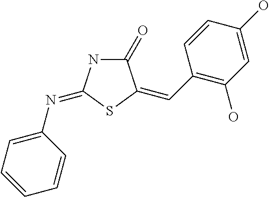

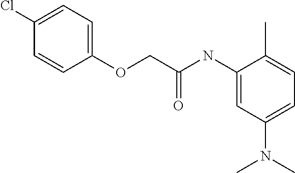

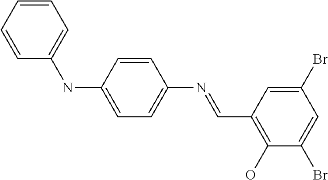

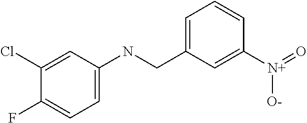

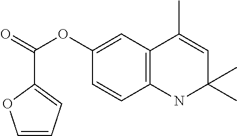

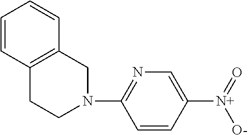

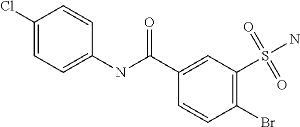

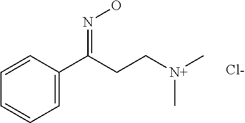

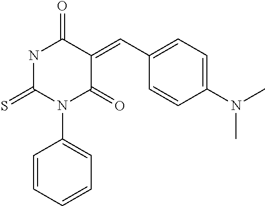

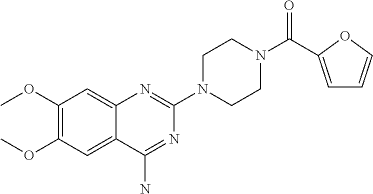

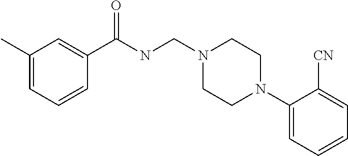

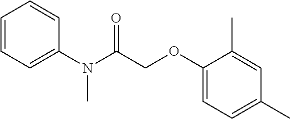

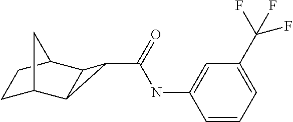

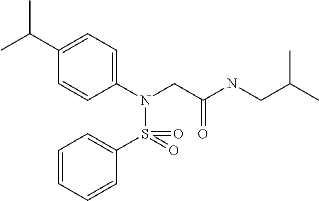

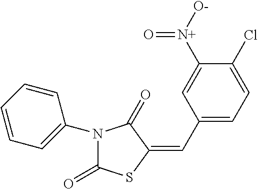

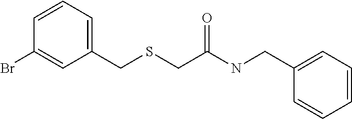

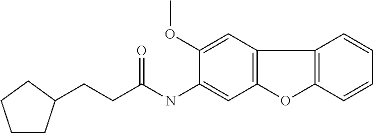

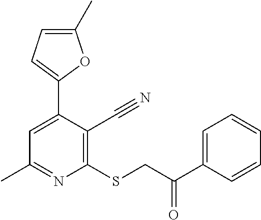

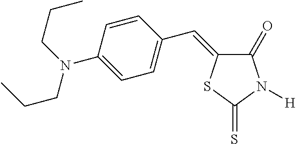

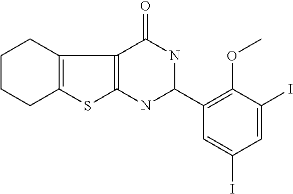

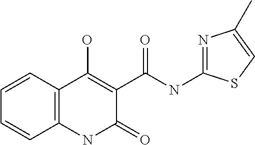

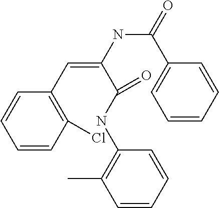

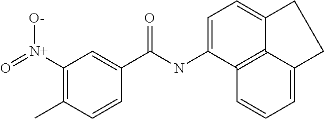

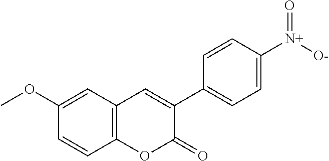

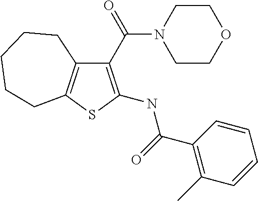

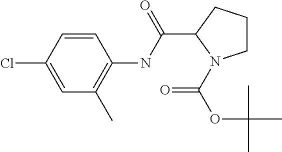

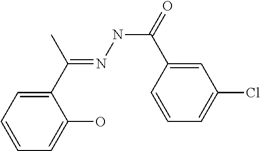

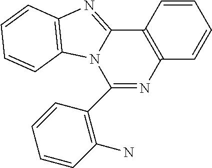

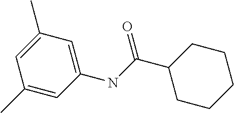

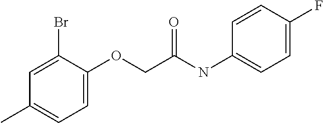

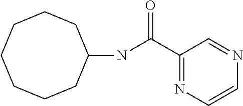

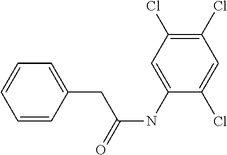

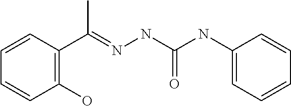

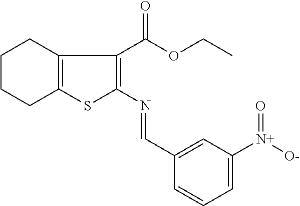

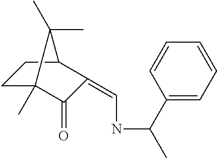

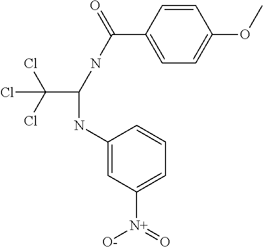

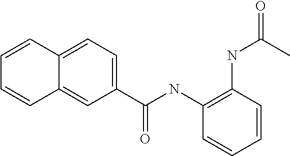

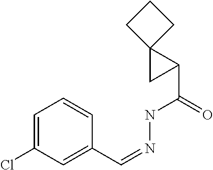

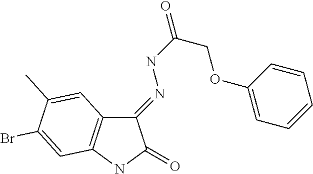

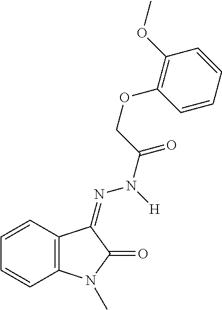

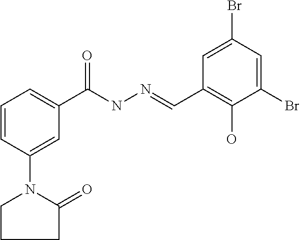

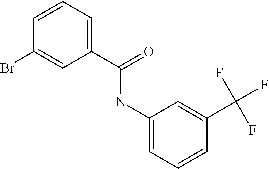

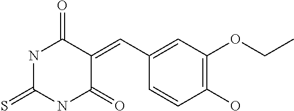

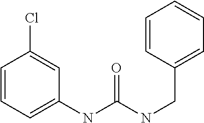

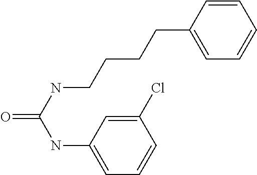

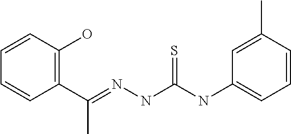

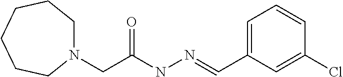

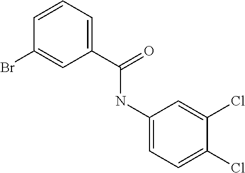

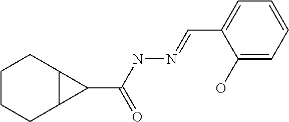

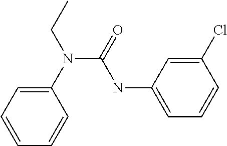

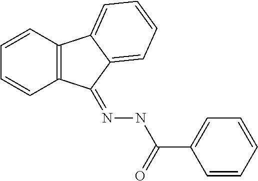

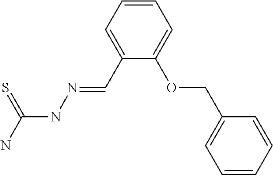

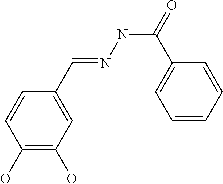

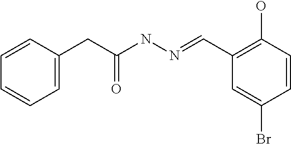

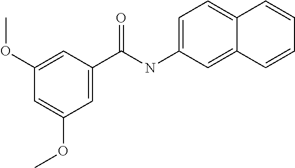

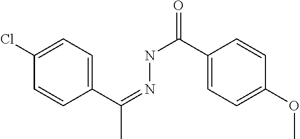

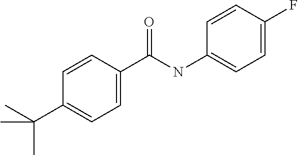

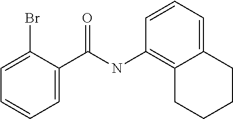

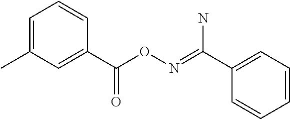

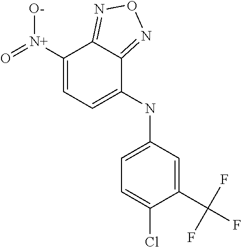

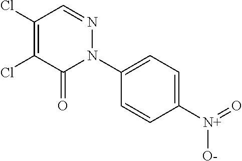

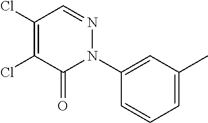

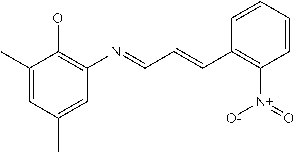

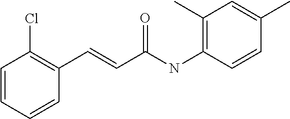

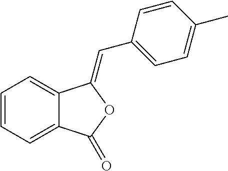

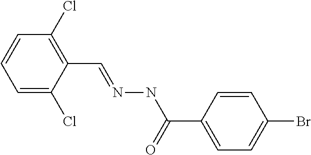

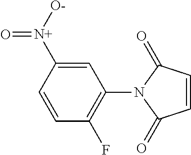

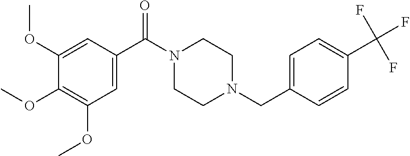

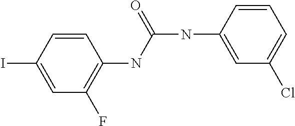

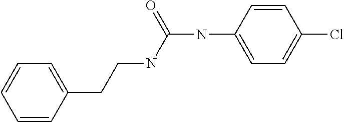

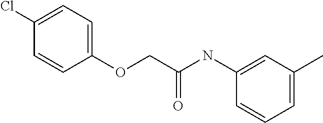

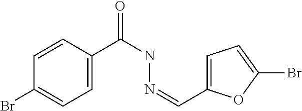

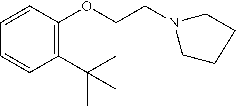

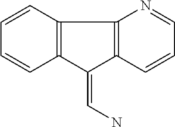

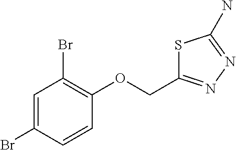

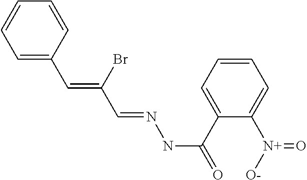

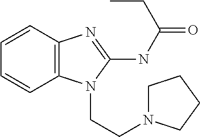

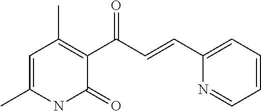

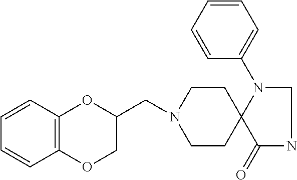

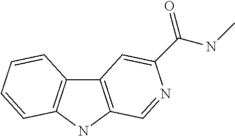

### Screening Methods and Assays

### Assays for Modulators of KCNQ Potassium Channels

Voltage-gated potassium channels are important regulatory proteins for controlling electrical excitability. One common feature of these proteins is the tight coupling of channel activity to transient changes in transmembrane potential. Hence, those channels with sensitivity to membrane potential changes close to the resting potential have a critical role in controlling membrane excitability. One group of low threshold non-inactivating potassium channels is characterized by potent inhibition upon activation of muscarinic receptors, hence also known as “M channels”. Their biophysical properties together with specific protein subcellular localization enable powerful control of the firing rate of a neuron. Because M channels are also inhibited by the neurotransmitter-activated G-protein coupled signaling pathway, the resultant down-regulation of M current could also potentiate membrane excitability. These opposing regulatory mechanisms are an important aspect of M-current physiology.

To identify compounds with agonistic activities for M currents, stable cell lines for both KCNQ2 and KCNQ2/3 were generated. Under a pre-calibrated condition of medium-level rubidium efflux responses, more than 20,000 small molecule compounds were screened at a concentration of 10 μM. The selected condition allowed for identification of both inhibitors and potentiators. Selected compounds either increased or decreased the rubidium responses by more than 15% when compared to the control. A subset of the compounds were selected and validated for both potency and specificity. Among them, ZnPy displayed unusual potency in elevating KCNQ activities as judged by the non-radioactive rubidium efflux assay.

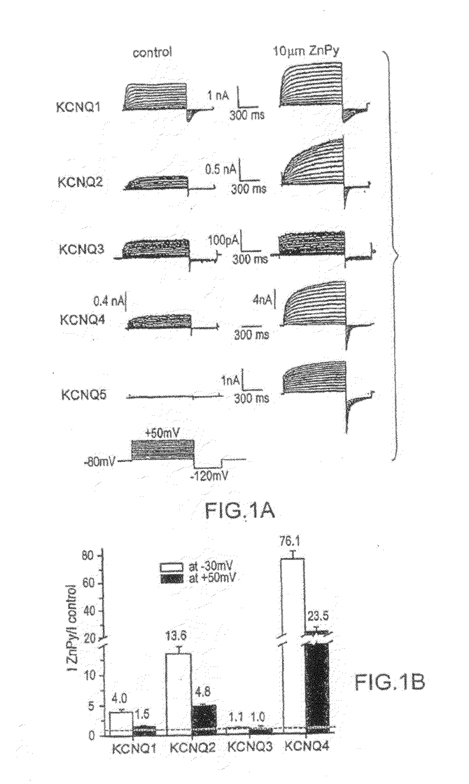

Protein sequences of KCNQ1 to KCNQ5 share considerable sequence homology. To determine target specificity of ZnPy, KCNQ1 to KCNQ5 in Chinese hamster ovary (CHO) cells were individually expressed and then tested for their sensitivity to ZnPy using a whole cell voltage clamp technique. Except for KCNQ3, the activities of all other four subtypes were potentiated by extracellular treatment with 10 μM ZnPy (FIG. 1A). KCNQ5 had little or no current under the expression condition used in the current studies. Surprisingly, ample KCNQ5 proteins were present on cell surface and became conductive upon ZnPy treatment. The observed potentiation was rapidly reversible and was not accompanied by any significant changes in membrane capacitance. Thus, regulation of channel density by trafficking could not be the major mechanism responsible for the potentiation. FIG. 1B shows that, depending on the specific channel, a 4 to 76-fold increase at −30 mV and a 1.5 to 24-fold increase at +50 mV (n≧4, p<0.001) were observed. In additional tests of other channels, it was found that ZnPy did not potentiate channel activities of human ether-a-go-go related gene (hERG), Kv2.1 delay rectifier voltage-gated potassium channel, Kv4.2 A-type potassium channel, or N-type calcium channel. These results are evidence for target-specific potentiation.

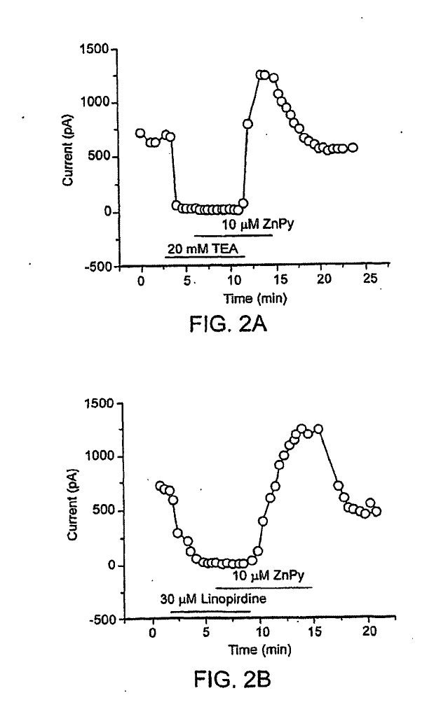

To be more definitive that the effects were exerted on potassium conductance, current sensitivity to TEA was examined, which is a common inhibitor for potassium channels. FIG. 2A shows that KCNQ2 currents were completely blocked by 20 mM external TEA. Extracellular application of 10 μM ZnPy was unable to reverse the TEA inhibition. However, the removal of TEA allowed for immediate recovery of ionic current and a continuation of potentiation above the initial amplitude, presumably by ZnPy. Indeed, subsequent removal of ZnPy reversed the current to the initial amplitude (FIG. 2A). Linopirdine inhibits both native M currents and recombinantly expressed KCNQ channels. Indeed, 30 μM linopirdine caused a rapid inhibition of KCNQ2 current in CHO cells. Similar to the TEA treatment above, continued perfusion of ZnPy induced a significant potentiation after removal of linopirdine (FIG. 2B). Thus, ZnPy acts on KCNQ-induced potassium current.

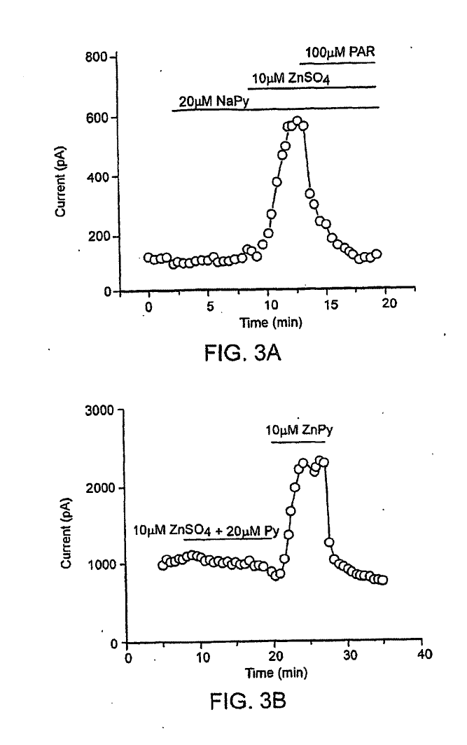

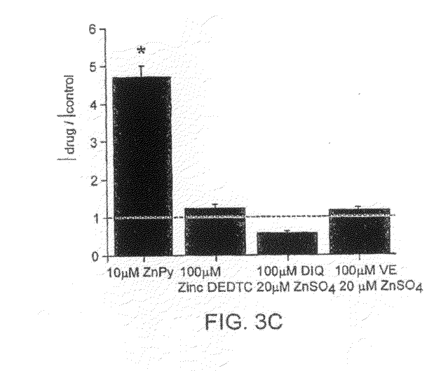

To determine whether Zn²⁺ and/or pyrithione are the causal factor(s) for the potentiation, the effects were tested using a CHO cell line expressing KCNQ2 by whole cell voltage clamp. Sodium pyrithione is water soluble, and ionizes at pH 7.8. FIG. 3A shows that no change in current amplitude was observed at 20 μM sodium pyrithione. Supplementation of 10 μM ZnSO₄ to the perfusion, which allows for the formation of ZnPy, induced a marked increase in current amplitude. The potentiation was abolished by 4-(2)-(pyridylazo)resorcinol (PAR), a potent zinc chelator. PAR itself had no effect on KCNQ2 channel activity. Furthermore, substitution of ZnSO₄ with CuSO₄ or CdCl₂ in the above experiments did not induce any potentiation. Hence, zinc and ionized pyrithione, either as a mixture of two independent entities or in a complexed form, are sufficient in conferring potentiation. To determine whether the two chemical components, zinc and pyrithione, might function independently but in a mixture, the formation of ZnPy from zinc salts and non-ionized pyrithione requires incubation at 90° C. in the absence of a catalyst. FIG. 3B shows that such a mixture without a prior incubation at 90° C. had no effects on the current amplitude. These results support that ZnPy, only as a complexed form, is causal to the potentiation.

ZnPy is an organozinc compound and an ionophore. To distinguish potential chemical interaction with the channel proteins from ionophore effects, modulatory effects by three additional zinc ionophores were tested: zinc diethyldithiocarbamate (DEDTC), 5,7-diiodo-8-hydroxyquinoline (DIQ) and (±)-α-tocopherol (VE). None displayed any potentiation effect on current amplitude (FIG. 3C), indicative that the ionophore effect alone is not sufficient for the observed potentiation.

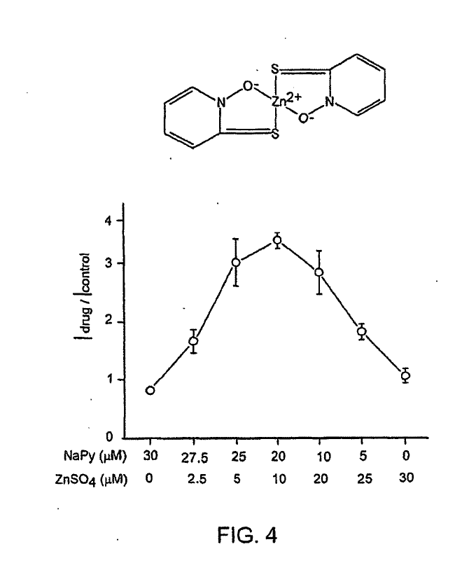

ZnPy, if causal to the potentiation, should have a preferential stoichiometry. Based on crystallographic studies and elemental analyses, ZnPy is a complex of one zinc atom chelated by two pyrithione units by way of sulfur and oxygen atoms (FIG. 4, upper panel).

Because organic solvents were used in these earlier studies, the optimal stoichiometry for potentiation of KCNQ channels in aqueous solution was examined. FIG. 4 shows the effects of potentiation induced by different ratios of sodium pyrithione and ZnSO₄. At a constant combined final concentration of 30 μM, the maximal potentiation was observed at a 2:1 ratio mixture of sodium pyrithione to ZnSO₄. This ratio of mixture is chemically comparable to that of 10 μM ZnPy. Indeed, the degree of potentiation is significant but slightly less that of 10 μM preformed ZnPy (FIG. 1B). Together, these results are in agreement with the notion that the potentiation caused by interaction with the KCNQ channel protein is dependent on specific stoichiometry of ZnPy.

The activity of a potassium channel comprising a KCNQ polypeptide can be assessed using a variety of in vitro and in vivo assays. Preferably, the in vivo assays disclosed herein in the example section are used to identify KCNQ openers for treatment of pain. Such assays are used to test for inhibitors and activators of KCNQ channels, for the identification of compounds that reduce pain in a subject. Assays for modulatory compounds include, e.g., measuring current; measuring membrane potential; measuring ion flux; e.g., potassium or rubidium; measuring potassium concentration; measuring second messengers and transcription levels, using potassium-dependent yeast growth assays; measuring pain responses in mice, e.g., with formalin algesia or hotplate assays; measuring ligand binding; and using, e.g., voltage-sensitive dyes, radioactive tracers, and patch-clamp electrophysiology.

Modulators of the potassium channels are tested using biologically active KCNQ channels, either recombinant or naturally occurring. KCNQ channels, preferably human KCNQ channels, can be isolated in vitro, co-expressed or expressed in a cell, or expressed in a membrane derived from a cell. In such assays, a KCNQ polypeptide is expressed alone to form a homomeric potassium channel or is co-expressed with a second alpha subunit (e.g., another KCNQ family member) so as to form a heteromeric potassium channel. A KCNQ channel can also be expressed with additional beta subunits. Samples or assays that are treated with a potential potassium channel inhibitor or activator are compared to control samples without the test compound, to examine the extent of modulation. Control samples (untreated with activators or inhibitors) are assigned a relative potassium channel activity value of 100. Inhibition of KCNQ channels is achieved when the potassium channel activity value relative to the control is about 90%, preferably 50%, more preferably 0-25%. Activation of KCNQ channels is achieved when the potassium channel activity value relative to the control is 110%, more preferably 150%, more preferably 200-500% higher, preferably 1000% or higher. Compounds that increase the flux of ions will cause a detectable increase in the ion current density by increasing the probability of a KCNQ channel being open, by decreasing the probability of it being closed, by increasing conductance through the channel, and/or by allowing the passage of ions.

Changes in ion flux may be assessed by determining changes in polarization (i.e., electrical potential) of the cell or membrane expressing the KCNQ potassium channel. A preferred means to determine changes in cellular polarization is by measuring changes in current (thereby measuring changes in polarization) with voltage-clamp and patch-clamp techniques, e.g., the “cell-attached” mode, the “inside-out” mode, and the “whole cell” mode (see, e.g., Ackerman et al., New Engl. J. Med. 336:1575-1595 (1997)). Whole cell currents are conveniently determined using the standard methodology (see, e.g., Hamil et al., PFlugers. Archiv. 391:85 (1981). Other known assays include: radiolabeled rubidium flux assays and fluorescence assays using voltage-sensitive dyes (see, e.g., Vestergarrd-Bogind et al., J. Membrane Biol. 88:67-75 (1988); Daniel et al., J. Pharmacol. Meth. 25:185-193 (1991); Holevinsky et al., J. Membrane Biology 137:59-70 (1994)). Assays for compounds capable of inhibiting or increasing potassium flux through the channel proteins can be performed by application of the compounds to a bath solution in contact with and comprising cells having a channel of the present invention (see, e.g., Blatz et al., Nature 323:718-720 (1986); Park, J. Physiol. 481:555-570 (1994)). Generally, the compounds to be tested are present in the range from 1 pM to 100 mM. Cells expressing the channel can express recombinant KCNQ (e.g., CHO cells or Xenopus cells) or endogenous KCNQ channels (e.g., rat dorsal root ganglion).

The effects of the test compounds upon the function of the channels can be measured by changes in the electrical currents or ionic flux or by the consequences of changes in currents and flux. Changes in electrical current or ionic flux are measured by either increases or decreases in flux of ions such as potassium or rubidium ions. The cations can be measured in a variety of standard ways. They can be measured directly by concentration changes of the ions or indirectly by membrane potential or by radio-labeling of the ions. Consequences of the test compound on ion flux can be quite varied. Accordingly, any suitable physiological change can be used to assess the influence of a test compound on the channels of this invention. The effects of a test compound can be measured by a toxin binding assay. When the functional consequences are determined using intact cells or animals, one can also measure a variety of effects such as transmitter release (e.g., dopamine), hormone release (e.g., insulin), transcriptional changes to both known and uncharacterized genetic markers (e.g., northern blots), cell volume changes (e.g., in red blood cells), immunoresponses (e.g., T cell activation), changes in cell metabolism such as cell growth or pH changes, and changes in intracellular second messengers such as Ca²⁺, or cyclic nucleotides. KCNQ orthologs will generally confer substantially similar properties on a channel comprising a KCNQ polypeptide, as described above. Preferably human KCNQ channels are used in the assays of the invention. Optionally, KCNQ orthologs from other species such as rat or mouse, preferably a mammalian species, are used in the assays of the invention.

### Compounds and Screening Methods

According to the present invention, any compound capable of binding zinc with moderate affinity and having sufficient lipophilic properties and one or more compatible interaction structural moiety is capable of effecting the treatment and protection demonstrated in the present invention with e.g., zinc-pyrithione. It is a fungicide and bactericide at high concentrations. It is highly lipophilic and therefore penetrates membranes easily. This permits zinc pyrithione to transport zinc across cell membranes, and is therefore considered a zinc ionophore.

In addition to zinc-pyrithione, another group of zinc ionophores, the dithiocarbamates, can treat pain in accordance with the present invention. The following are examples of compounds which have been shown in accordance with the present invention to possess zinc-ionophore properties: zinc pyrithione, the heterocyclic amines including, for example, 5,7-Diiodo-8-hydroxyquinoline, and 8-Hydroxyquinoline; the dithiocarbamates including, for example, pyrrolidine dithiocarbamate and diethyldithiocarbamate, disulfiram and dimethyldithiocarbamate; and Vitamins including, but not limited to, Vitamin E and Vitamin A. Properties associated with zinc ionophores include, but are not limited to, an ability to alter cytosolic PKC-content and an ability to alter the nuclear activity of transcription factors NF-kB, AP-1 and Spl. According to the present invention zinc-pyrithione may operate at the cell signalling level, as demonstrated by its ability to alter cytosolic PKC-content. Further, according to the present invention, zinc-pyrithione may operate at the transcriptional level, as demonstrated by its ability to alter the nuclear activity of transcription factors NF-kB, AP-1 and Spl. Still further, according to the present invention zinc-pyrithione can upregulate cytoprotective proteins, for example HSP70.

According to the present invention, any compound capable of binding zinc, through a direct bond, a coordination interaction, or a chelate interaction, with moderate affinity is capable of effecting the treatment and protection demonstrated in the present invention with e.g., zinc-pyrithione. In certain instances, such compounds have sufficient lipophilic properties to penetrate cell membranes. The following are examples of compounds which have been shown in accordance with the present invention to possess zinc-ionophore properties: zinc pyrithione, the heterocyclic amines including, for example, 5,7-Diiodo-8-hydroxy quinoline, and 8-Hydroxyquinoline; the dithiocarbamates including, for example, pyrrolidine dithiocarbamate and diethyldithiocarbamate, disulfiram and dimethyldithio carbamate; and Vitamins including, but not limited to, Vitamin E and Vitamin A. Properties associated with zinc ionophores include, but are not limited to, an ability to alter cytosolic PKC-α content and an ability to alter the nuclear activity of transcription factors NF-kB, AP-1 and Spl. According to the present invention zinc-pyrithione may operate at the cell signalling level, as demonstrated by its ability to alter cytosolic PKC-α content. Further, according to the present invention, zinc-pyrithione may operate at the transcriptional level, as demonstrated by its ability to alter the nuclear activity of transcription factors NF-kB, AP-1 and Spl. Still further, according to the present invention zinc-pyrithione may upregulate cytoprotective proteins, for example HSP70.

Chemical compounds which increase ion flux through KCNQ potassium channels, are made according to methodology well known to those of skill in the art.

The compounds tested as modulators of KCNQ channels can be any small chemical compound, or a biological entity, such as a protein, sugar, nucleic acid or lipid. Typically, test compounds will be small chemical molecules and peptides. Essentially any chemical compound can be used as a potential modulator or ligand in the assays of the invention, although most often compounds can be dissolved in aqueous or organic solutions are used. The assays are designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Bucks Switzerland) and the like.

In one embodiment, high throughput screening methods involve providing a combinatorial chemical or peptide library containing a large number of potential therapeutic compounds (potential modulator or ligand compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

In one embodiment, the invention provides solid phase based in vitro assays in a high throughput format, where the cell or tissue expressing a KCNQ channel is attached to a solid phase substrate. In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microliter plate can assay about 96 modulators. If 1536 well plates are used, then a single plate can easily assay from about 100 to about 1500 different compounds. It is possible to assay several different plates per day; assay screens for up to about 6,000-20,000 different compounds is possible using the integrated systems of the invention.

Compounds of the present invention can be prepared using readily available starting materials or known intermediates. One of skill in the art will recognize that other compounds of the present invention can be prepared from known intermediates using reactions and techniques known to those ordinary skill in the art.

Methods for preparing dimers, trimers and higher homologs of small organic molecules, such as those of the present invention, as well as methods of functionalizing a polyfunctional framework molecule are well known to those of skill in the art.

According to the present invention, small concentrations of a zinc ionophore in the nanomolar and picomolar range, such as from about 10 pM to about 1 μM can alleviate pain. In another embodiment small concentrations of a zinc ionophore in the nanomolar and picomolar range, such as from about 10 pM to about 1 μM can regulate gene expression by modulating the activity of transcription factors in the various organ systems, including but not limited to the brain and heart of mammals, including humans. Transcription factors which may be modulated in accordance with the present invention include, but are not limited to NF-kB, AP-1 and Spl.

Thus, according to the present invention the concentration of zinc ionophore used to treat pain ranges from about 0.005 μg zinc ionophore per kg of body weight to about 200 mg zinc ionophore per kg of body weight. In a further embodiment of the present invention the concentration of zinc ionophore used to treat pain ranges from about 1.0 μg zinc ionophore per kg of body weight to about 800 μg zinc ionophore per kg of body weight.

Certain compounds of the present invention possess asymmetric carbon atoms (optical centers) or double bonds; the racemates, diastereomers, geometric isomers and individual isomers are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

### Solid State and Soluble High Throughput Assays

In one embodiment the invention provide soluble assays using potassium channels comprising KCNQ; a membrane comprising a KCNQ potassium channel, or a cell or tissue expressing potassium channels comprising KCNQ, either naturally occurring or recombinant. In another embodiment, the invention provides solid phase based in vitro assays in a high throughput format, where KCNQ potassium channel attached to a solid phase substrate.

In the high throughput assays of the invention, it is possible to screen up to several thousand different modulators or ligands in a single day. In particular, each well of a microtiter plate can be used to run a separate assay against a selected potential modulator, or, if concentration or incubation time effects are to be observed, every 5-10 wells can test a single modulator. Thus, a single standard microtiter plate can assay about 100 (e.g., 96) modulators. If 1536 well plates are used, then a single plate can easily assay from about 100-about 1500 different compounds. It is possible to assay many plates per day; assay screens for up to about 6,000, 20,000, 50,000, or more than 100,000 different compounds is possible using the integrated systems of the invention.

The channel of interest, or a cell or membrane comprising the channel of interest can be bound to the solid state component, directly or indirectly, via covalent or non covalent linkage e.g., via a tag. The tag can be any of a variety of components. In general, a molecule which binds the tag (a tag binder) is fixed to a solid support, and the tagged molecule of interest is attached to the solid support by interaction of the tag and the tag binder.

A number of tags and tag binders can be used, based upon known molecular interactions well described in the literature. For example, where a tag has a natural binder, for example, biotin, protein A, or protein G, it can be used in conjunction with appropriate tag binders (avidin, streptavidin, neutravidin, the Fc region of an immunoglobulin, etc.) Antibodies to molecules with natural binders such as biotin are also widely available and appropriate tag binders; see, SIGMA Immunochemicals 1998 catalogue SIGMA, St. Louis Mo.).

Similarly, any haptenic or antigenic compound can be used in combination with an appropriate antibody to form a tag/tag binder pair. Thousands of specific antibodies are commercially available and many additional antibodies are described in the literature. For example, in one common configuration, the tag is a first antibody and the tag binder is a second antibody which recognizes the first antibody. In addition to antibody-antigen interactions, receptor-ligand interactions are also appropriate as tag and tag-binder pairs. For example, agonists and antagonists of cell membrane receptors (e.g., cell receptor-ligand interactions such as transferrin, c-kit, viral receptor ligands, cytokine receptors, chemokine receptors, interleukin receptors, immunoglobulin receptors and antibodies, the cadherein family, the integrin family, the selectin family, and the like; see, e.g., Pigott & Power, The Adhesion Molecule Facts Book I (1993). Similarly, toxins and venoms, viral epitopes, hormones (e.g., opiates, steroids, etc.), intracellular receptors (e.g. which mediate the effects of various small ligands, including steroids, thyroid hormone, retinoids and vitamin D; peptides), drugs, lectins, sugars, nucleic acids (both linear and cyclic polymer configurations), oligosaccharides, proteins, phospholipids and antibodies can all interact with various cell receptors.

Synthetic polymers, such as polyurethanes, polyesters, polycarbonates, polyureas, polyamides, polyethyleneimines, polyarylene sulfides, polysiloxanes, polyimides, and polyacetates can also form an appropriate tag or tag binder. Many other tag/tag binder pairs are also useful in assay systems described herein, as would be apparent to one of skill in the art.

Common linkers such as peptides, polyethers, and the like can also serve as tags, and include polypeptide sequences, such as poly gly sequences of between about 5 and 200 amino acids. Such flexible linkers are known to persons of skill in the art. For example, poly(ethylene glycol) linkers are available from Shearwater Polymers, Inc. Huntsville, Ala. These linkers optionally have amide linkages, sulfhydryl linkages, or heterofunctional linkages.

Tag binders are fixed to solid substrates using any of a variety of methods currently available. Solid substrates are commonly derivatized or functionalized by exposing all or a portion of the substrate to a chemical reagent which fixes a chemical group to the surface which is reactive with a portion of the tag binder. For example, groups which are suitable for attachment to a longer chain portion would include amines, hydroxyl, thiol, and carboxyl groups. Aminoalkylsilanes and hydroxyalkylsilanes can be used to functionalize a variety of surfaces, such as glass surfaces. The construction of such solid phase biopolymer arrays is well described in the literature. See, e.g., Merrifield, J. Am. Chem. Soc. 85:2149 2154 (1963) (describing solid phase synthesis of, e.g., peptides); Geysen et al., J. Immun. Meth. 102:259 274 (1987) (describing synthesis of solid phase components on pins); Frank & Doring, Tetrahedron 44:60316040 (1988) (describing synthesis of various peptide sequences on cellulose disks); Fodor et al., Science, 251:767 777 (1991); Sheldon et al., Clinical Chemistry 39(4):718 719 (1993); and Kozal et al., Nature Medicine 2(7):753759 (1996) (all describing arrays of biopolymers fixed to solid substrates). Non-chemical approaches for fixing tag binders to substrates include other common methods, such as heat, cross-linking by UV radiation, and the like.

### Mechanism

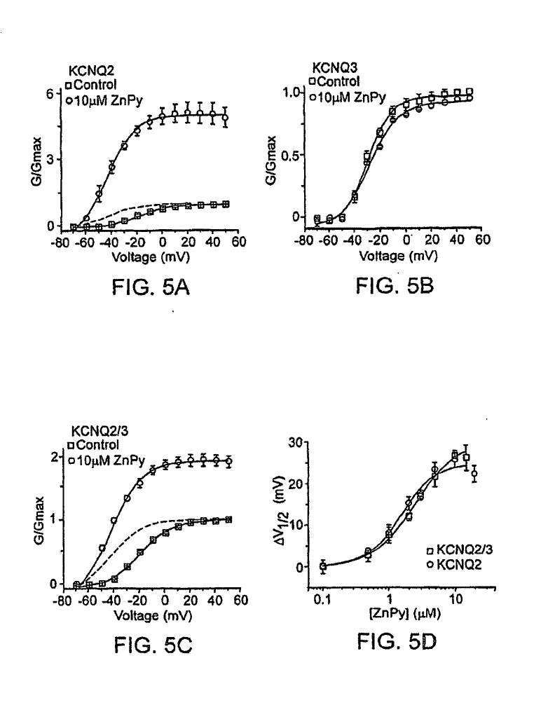

To study mechanisms for the potentiation, voltage-dependent activation was examined using heterologously expressed KCNQ2 and KCNQ3 channels. In the presence of 10 μM ZnPy, the V_{1/2} (voltage required for half maximal activation) of KCNQ2 was left-shifted by as much as 25.5±1.5 mV, from −18.9±0.6 mV to −44.4±0.9 mV (n≧4, p<0.001). In contrast, KCNQ3, which did not show any current potentiation by ZnPy (FIG. 1A), also displayed no change on V_{1/2} (FIGS. 5, A & B). Heteromultimeric assembly of KCNQ2 and KCNQ3 channels is thought to be the molecular constituent for M-current. ZnPy consistently caused a hyperpolarizing shift of V_{1/2} of KCNQ2/3 channels by 24.4±0.8 mV, from −20.3±0.8 mV to −44.7±1.9 mV (n≧4, p<0.001) (FIG. 5C). The effect of ZnPy was dose-dependent and displayed an EC₅₀ value of 1.5±0.3 μM for KCNQ2 and 2.4±0.4 μM for KCNQ2/3 (FIG. 5D). This supports a “dominant” role for KCNQ2 in conferring sensitivity to ZnPy. The dosage dependent curve was fitted with a Hill equation with a coefficient of 1.8 for KCNQ2 and 1.4 for KCNQ2/3.

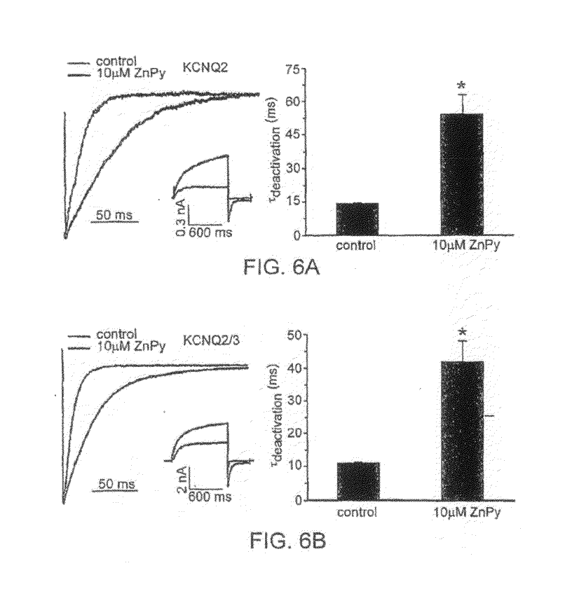

In addition to the hyperpolarizing shift of V_{1/2}, ZnPy markedly reduced both activation and deactivation rates. Specifically, the activation time constant of KCNQ2/3 at +50 mV changed from 75.6±4.9 to 157.8±23.5 milliseconds (n=6, p<0.001), similarly from 163.5±13.7 to 293.1±11.2 milliseconds (n=5, p<0.001) for KCNQ2. The deactivation time constant for KCNQ2/3 at −120 mV increased from 12.4±0.7 to 41.9±6.4 milliseconds (n=8, p<0.001) and from 14.1±0.6 to 54.4±0.9 milliseconds (n=4, p<0.001) for KCNQ2 (FIG. 6). The combination of increase of maximal conductance (Gₘₐₓ) and hyperpolarizing shift of half maximal activation contributes to the overall augmentation of current amplitude.

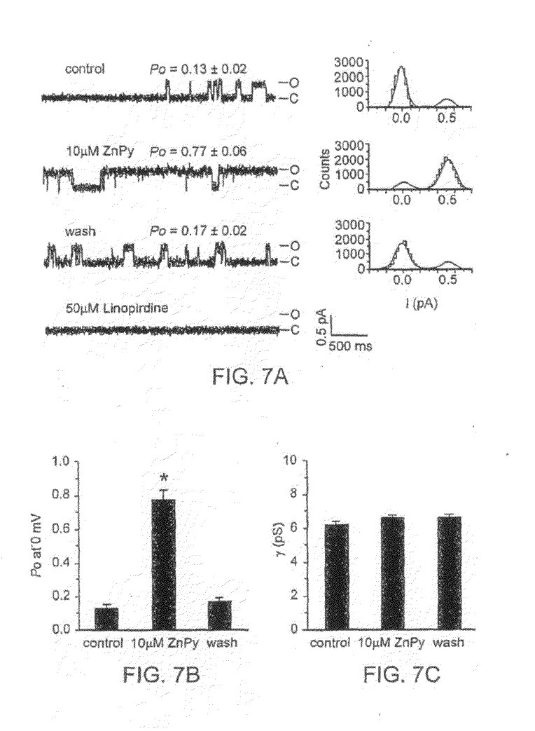

To be more definitive about the mechanism of Gₘₐₓ augmentation by ZnPy, single channel activities of KCNQ2 at 0 mV saturated voltage were recorded. FIG. 7A shows a significant increase of single channel open probability (Pₒ) from 0.13±0.02 to 0.77±0.06 (n=5, p<0.001) in the presence of 10 μM ZnPy. The single channel activity was fully blocked by linopirdine. Consistent with macroscopic measurements, the ZnPy effects were full reversible (FIG. 7B) and displayed consistent single channel conductance of 6.6±0.15 pS (n=5) in the presence or absence of ZnPy (FIG. 7C). The 5.9-fold increase in Pₒ in the presence of 10 μM ZnPy could fully account for the overall augmentation of Gₘₐₓ.

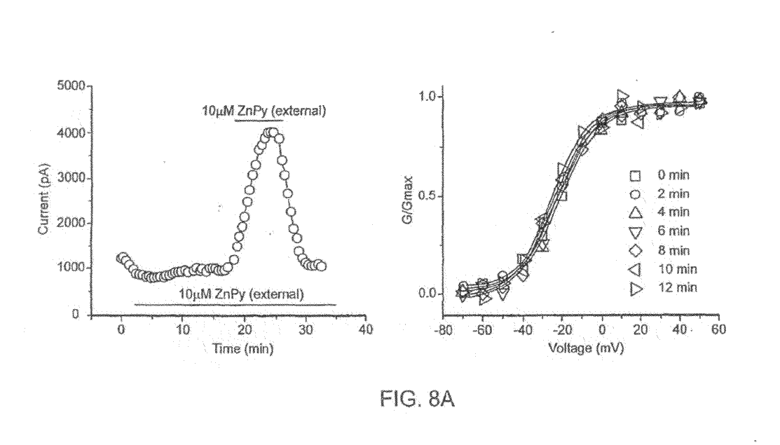

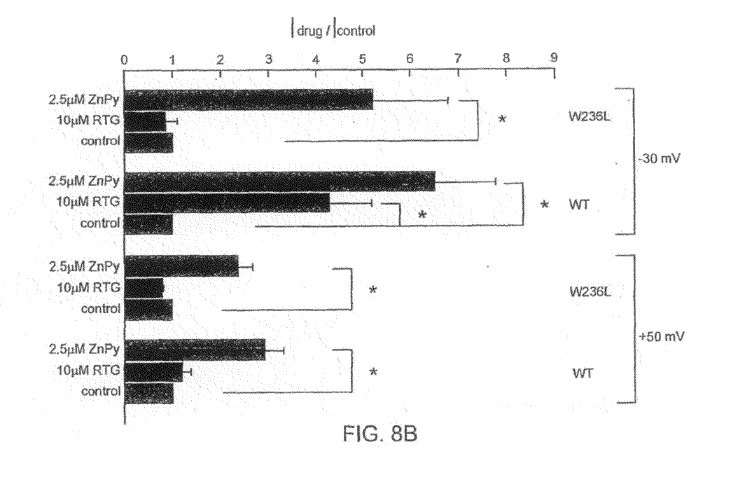

The effect by ZnPy was consistently observed by external application. To determine whether the effect could be achieved through intracellular application, KCNQ current was recorded using a pipette solution lacking EGTA but supplemented with 10 μM of ZnPy (FIG. 8A, left). The channel activities were monitored over periods of more than 10 minutes, a condition previously confirmed to be sufficient for intracellular diffusion. ZnPy when applied intracellularly induced no potentiation, either with or without EGTA, which chelates Zn²⁺. Under this condition, when ZnPy was applied externally, the potentiation was fully inducible (FIG. 8A, left). To be more definitive, V_{1/2} values were measured at different times after establishing a stable seal and internal perfusion of ZnPy through a recording pipette. Indeed, the V_{1/2} was consistent and identical to that of KCNQ2 control (FIG. 8A, right panel). This provides the evidence for external accessibility of ZnPy for KCNQ interactions.

Retigabine (RTG) binding to KCNQ channels causes V_{1/2} shift to a more hyperpolarizing potential but with little change in channel conductance. In addition, it is most effective on KCNQ3 and essentially has no effect on KCNQ1. These features represent a major distinction between retigabine and ZnPy. The KCNQ2 (W236L) mutant is a channel fully functional but no longer sensitive to RTG. When treated with ZnPy, this mutant has comparable sensitivity to KCNQ2 wild type at either −30 mV or +50 mV (FIG. 8B). Together, these observations suggest that ZnPy acts on a new site in causing its agonistic effects.

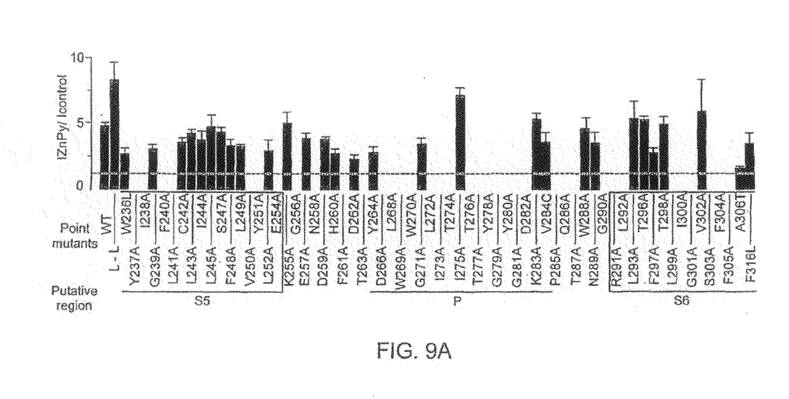

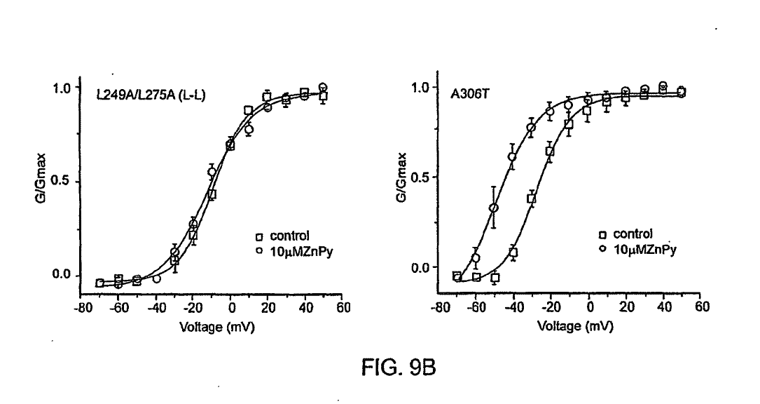

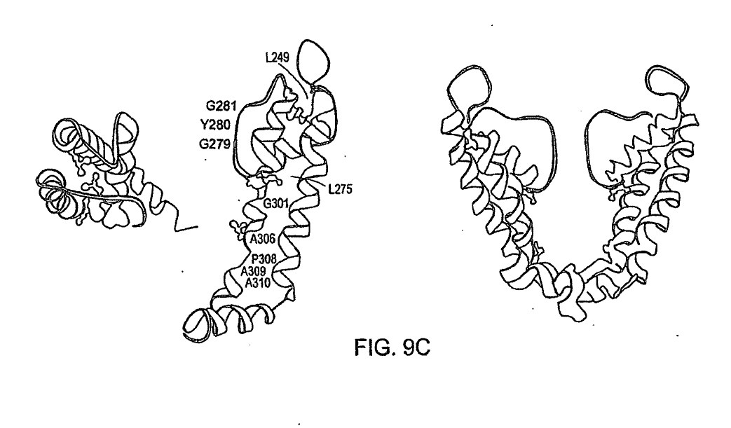

Alteration of V_{1/2} and maximal conductance (Gₘₐₓ) is consistent with a role of ZnPy interacting with the gating machinery. ZnPy appears to access from the extracellular side, and many key residues of gating machinery lie between S5 to S6. To investigate the molecular determinants responsible for ZnPy effects, a series of site-directed mutants was constructed by systematically substituting residues with Ala covering and flanking the pore region. FIG. 9A shows those KCNQ2 mutants that are functional and their levels of sensitivity to ZnPy displayed by number of folds of current increase at +50 mV induced. Examining both V_{1/2} and folds of increase revealed several positions contributing to ZnPy sensitivity. Of particular significance, both L249A and L275A mutants have significant reduction on changes in V_{1/2}. The double mutation of both leucine residues KCNQ2(L249A/L275A) completely abolished the V_{1/2} shift caused by ZnPy while it displayed 8.6±2.2 (n=3, p<0.001) folds of current increase (FIGS. 9, A & B). In contrast, KCNQ2(A306T) showed little potentiation in Gₘₐₓ by ZnPy but remained sensitive to ZnPy, displaying the hyperpolarizing shift by 24.1±3.1 mV (n=6, p<0.001) (FIG. 9B). Together, evidence from investigating these mutants begins to define molecular determinants critical for the ZnPy effects. In addition, the two effects are separable. These residues lie in the critical regions of channel protein consistent with a role in stabilizing open conformation (FIG. 9C).

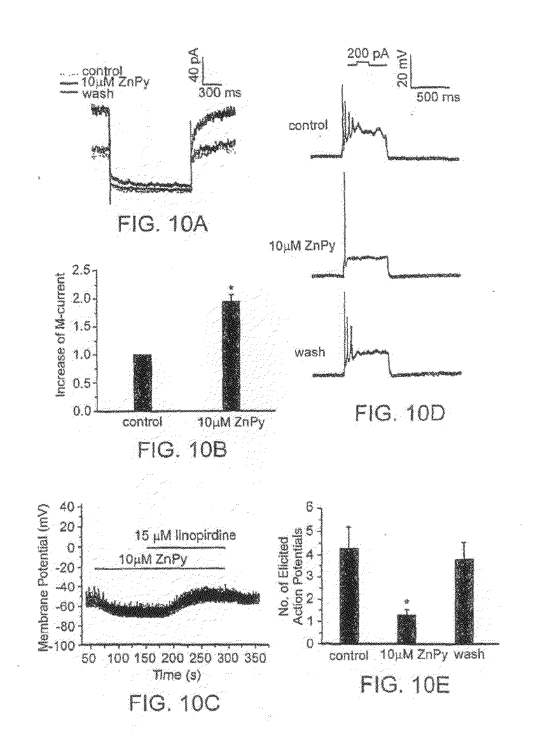

To examine any effect on native M-current, isolated neurons were recorded using a standard protocol. FIG. 10A shows a typical M-current recorded before and after addition of 10 μM ZnPy. Consistent with results using heterologously expressed KCNQ2/3 channels, ZnPy induced a significant potentiation of 1.9 fold (FIG. 10B). In CHO cells expressing the KCNQ2 channel, 10 μM ZnPy caused significant hyperpolarization. Similarly, in hippocampal neurons, treatment with 10 μM ZnPy resulted in noticeable hyperpolarization of membrane potential (FIG. 10C). Application of an M channel antagonist, linopirdine, completely abolished the hyperpolarization of membrane potential, consistent with the notion that ZnPy acts on M current. This result is also in agreement with earlier experiments where in the presence of linopirdine, ZnPy was unable to induce potentiation (FIG. 2B).

One of the major physiological functions of M-current is to suppress repetitive discharges in neurons. Persistence of repetitive firing by current injection was tested in the presence and absence of ZnPy. FIG. 10D shows the induced action potential spikes fired by an isolated hippocampal neuron upon current injection. Perfusion of ZnPy immediately abolished the repetitive firing. The activity was restored after the removal of ZnPy (FIG. 10D, middle and lower panels; and FIG. 10E). Together, the above series of experiments provides direct evidence that ZnPy agonistically modulates native M-current by a mechanism consistent with what has been characterized for recombinant KCNQ2/3 channels.

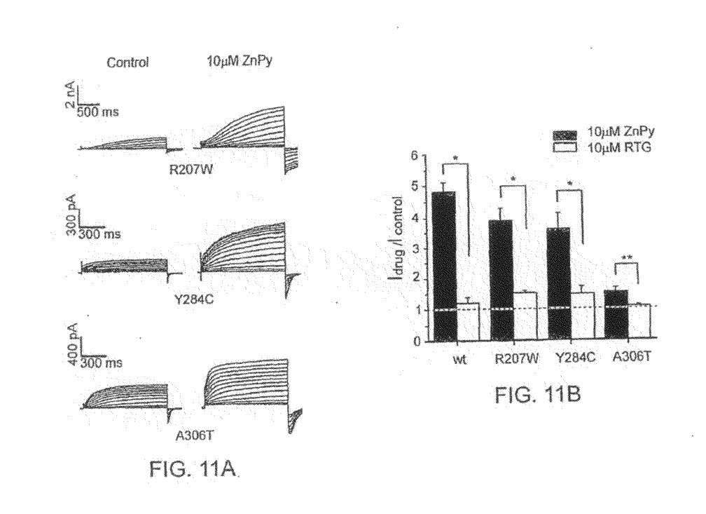

Mutations in KCNQ2 and KCNQ3 genes have been found in patients who suffer from BFNCs and myokymia. The common phenotype of these mutants is reduction of KCNQ currents as a result of decrease in either channel activities or protein expression on the cell surface. Because BFNC is autosomal dominant, M currents in patients will likely be heteromultimers of mutant and wildtype KCNQ2 and KCNQ3 subunits. ZnPy potently up-regulates the KCNQ channels across a broad range of membrane potentials. It was thus hypothesized that ZnPy could acutely potentiate channel activity of those mutants, thereby rescuing the channel activity. To test this notion, homomultimers of KCNQ2 were tested, because ZnPy is dominant in conferring sensitivity (FIG. 5C). Three human mutations, KCNQ2(R207W), KCNQ2(Y284C), and KCNQ2(A306T) were expressed, that have been previously shown to produce channel proteins with reduced conductivity. FIG. 11A shows the current traces recorded from cells transiently transfected with each mutant. The increase caused by ZnPy was more than 3-fold for both KCNQ2(R207W) and KCNQ2(Y284C) mutants (n=4, p<0.001). Consistently, ZnPy is less effective on KCNQ2 (A306T) indicating that the integrity of this region is required for the ZnPy effect. In contrast, retigabine induced less then 50% potentiation for all mutants (FIG. 11B). Ligand-mediated activation of KCNQ channels by ZnPy causes a significant hyperpolarization shift of voltage sensitivity and a marked reduction of the deactivation rate, resulting in channel opening across a wider range of membrane potential. Hence, in essence ZnPy effects confer a ligand-gating process in addition to voltage-gating for the KCNQ channels. The ZnPy-mediated opening may be observed under physiological resting potential causing hyperpolarization. These results provide experimental evidence that synthetic activators are capable of inducing activation of voltage-gated ion channels. ZnPy induced opening is reminiscent of the Ca²⁺ effect on calcium-activated potassium channels which are dually gated by membrane depolarization and intracellular Ca²⁺. At high concentration, Ca²⁺ can induce channel opening in the absence of membrane depolarization. Similarly, TRP channels (such as TRPM8) are sensitive to cold, menthol, voltage and PIP2. Menthol binding induced a significant hyperpolarizing shift of V_{1/2} from 32 to −31 mV.

Chemical openers for cation ion channels are very rare, especially considering the limited approaches available for identification. Using the reported approach and a non-radioactive rubidium efflux assay, a screening condition capable of identifying both agonistic and antagonistic modulators of potassium channels was optimized. Considering that agonistic activity is essentially a gain of function phenotype, the relatively large number of identified compounds with more than a 15% increase of Rb⁺ efflux activity suggests a robust readout range for capturing compounds with agonistic activities. Because almost all potassium channels are permeable to rubidium and many have already been tested by rubidium flux assay using radioactive ⁸⁶Rb⁺, it is conceivable that the screening strategy reported identifies agonists for different potassium channels.

The structure of ZnPy is unique when compared with other identified structures, such as retigabine that also displayed agonistic activity for KCNQ channels. The ability to activate the channel and to increase the overall conductance raises the question whether ZnPy could affect KCNQ channels via multiple different mechanisms including steady state expression on cell surface. Indeed, the KCNQ2 channel protein has a short half-life on the cell surface when compared to other potassium channels such as hERG (data not shown). ZnPy, like other identified compounds, could act in principle either chronically or acutely in potentiating channel activity. However, our evidence supports that the ZnPy effect on KCNQ2 and other KCNQ channels is mainly caused by an acute potentiation because of rapid and reversible effects in both heterologously expressed cells and cultured neurons (FIGS. 2, 3, 8 & 10).

The reported evidence is consistent with a model of specific interaction between pyrithione and KCNQ channel proteins. One hypothesis is that the pyrithione perhaps has low affinity, but is markedly enhanced when it is complexed with a zinc ion. The specific 1:2 zinc to pyrithione stoichiometry shown in FIG. 4 for the optimal potentiation raises the possibility that zinc plays a chaperonic role in “dimerizing” pyrithione, reminiscent of what has been observed for carbonic anhydrase II, where zinc appears to play a role in mediating a specific but reversible interaction critical for holoenzyme function. Indeed, a specific double mutant R362H/A419H in Shaker potassium channel displayed a strong hyperpolarizing shift of the G-V curve caused by Zn²⁺ binding. The overall conductance of the Shaker potassium channel, however, showed no change, supporting the idea that Zn²⁺ binding specifically stabilizes the activated state. The corresponding positions in KCNQ2 channel are R198 and K255, which cannot interact with Zn²⁺ directly. In addition, Zn²⁺ alone cannot induce any detectable effect (FIG. 3). This suggests that ZnPy confers the effects by a different molecular interaction.

ZnPy, in addition to a higher potency than retigabine, appears to exert KCNQ potentiation by a different molecular mechanism. Most noticeably, ZnPy is fully effective in potentiating the KCNQ2(W236L) mutant (FIG. 8B). In contrast, this mutant is no longer sensitive to retigabine. Furthermore, the ZnPy potentiates KCNQ1 but has no effect on KCNQ3 (FIGS. 1 & 5). On the contrary, retigabine is most effective on KCNQ3 but not KCNQ1. The effect of retigabine is mainly caused by a hyperpolarizing shift of voltage dependence. Hence, at saturated voltages such as +50 mV, retigabine could no longer induce additional effect on BFNC mutant channels, whereas ZnPy exerts a potent effect via increase in Gₘₐₓ (FIG. 11).

With respect to the molecular determinants critical for the ZnPy effects, the data suggest that L249, L275, and A306 are important residues to confer ZnPy sensitivity. Intriguingly, the L249A/L275A double mutant and the A306T mutant separately affect V_{1/2} and Gₘₐₓ (FIG. 9). If one assumes that KCNQ2 structure is similar to that of Kv1.2 within the transmembrane pore region, L249 and L275 will be on two separate α-helixes but facing the same side (FIG. 9C). The linear distance between these two residues is 10.6 Å consistent with interacting with ZnPy. Residue A306 is deeper inside the structure and positions between G301, the so-called gating hinge and the conserved putative Pro-Ala-Gly bend of the S6 domain. Both are critical components for voltage-mediated gating in KCNQ channels. Thus, these results indicate key residues required for ZnPy sensitivity. Their locations in channel structure are consistent with the notion that interaction with ZnPy causes stabilization of open conformation.

As a molecular probe, ZnPy has quite a unique feature both in its potency and mode of action. Because ZnPy potentiates KCNQ channels but not other voltage-gated potassium channels, it might be particularly useful for understanding the similarity and difference for these otherwise homologous potassium channels.

### Pharmaceutical Compositions

The term “pharmaceutically acceptable salts” or “pharmaceutically acceptable carrier” is meant to include salts of the active compounds which are prepared with relatively nontoxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al., Journal of Pharmaceutical Science 66:1-19 (1977)). Certain specific compounds of the present invention contain both basic and acidic functionalities that allow the compounds to be converted into either base or acid addition salts. Other pharmaceutically acceptable carriers known to those of skill in the art are suitable for the present invention.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the present invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

Certain compounds of the present invention can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

The invention also provides a pharmaceutical composition, comprising an effective amount a compound described herein and a pharmaceutically acceptable carrier. In an embodiment, compound is administered to the subject using a pharmaceutically-acceptable formulation, e.g., a pharmaceutically-acceptable formulation that provides sustained delivery of the compound to a subject for at least 12 hours, 24 hours, 36 hours, 48 hours, one week, two weeks, three weeks, or four weeks after the pharmaceutically-acceptable formulation is administered to the subject.

Actual dosage levels and time course of administration of the active ingredients in the pharmaceutical compositions of this invention may be varied so as to obtain an amount of the active ingredient which is effective to achieve the desired therapeutic response for a particular patient, composition, and mode of administration, without being toxic to the patient.

In use, at least one organozinc compound, according to the present invention is administered in a pharmaceutically effective amount to a subject in need thereof in a pharmaceutical carrier by intravenous, intramuscular, subcutaneous, or intracerebroventricular injection or by oral administration or topical application. In accordance with the present invention, one organozinc compound may be administered, preferably by the intravenous injection route, alone or in conjunction with a second, different zinc ionophore. By “in conjunction with” is meant together, substantially simultaneously or sequentially. In one embodiment, the organozinc compounds of the present invention, are administered acutely, such as, for example, substantially immediately following an injury that results in pain, such as surgery. The organozinc compound may therefore be administered for a short course of treatment, such as for about 1 day to about 1 week. In another embodiment, the organozinc compound of the present invention may be administered over a longer period of time to ameliorate chronic stress, such as, for example, for about one week to several months depending upon the condition to be treated.

By “pharmaceutically effective amount” as used herein is meant an amount of organozinc compound, e.g., zinc-pyrithione, high enough to significantly positively modify the condition to be treated but low enough to avoid serious side effects (at a reasonable benefit/risk ratio), within the scope of sound medical judgment. A pharmaceutically effective amount of zinc ionophore will vary with the particular goal to be achieved, the age and physical condition of the patient being treated, the severity of the underlying disease, the duration of treatment, the nature of concurrent therapy and the specific organozinc compound employed. For example, a therapeutically effective amount of a organozinc compound administered to a child or a neonate will be reduced proportionately in accordance with sound medical judgment. The effective amount of organozinc compound will thus be the minimum amount which will provide the desired anti-pain effect.

A decided practical advantage of the present invention is that the organozinc compound, e.g. zinc-pyrithione, may be administered in a convenient manner such as by the, intravenous, intramuscular, subcutaneous, oral or intracerebroventricular injection routes or by topical application, such as in eye drops or eye mist compositions. Depending on the route of administration, the active ingredients which comprise organozinc compound may be required to be coated in a material to protect the organozinc compound from the action of enzymes, acids and other natural conditions which may inactivate the organozinc compound. In order to administer organozinc compound by other than parenteral administration, the organozinc compound can be coated by, or administered with, a material to prevent inactivation. For example, the organozinc compound of the present invention may be co-administered with enzyme inhibitors or in liposomes. Enzyme inhibitors include pancreatic trypsin inhibitor, and trasylol. Liposomes include water-in-oil-in-water P40 emulsions as well as conventional and specifically designed liposomes.

The organozinc compound may be administered parenterally or intraperitoneally. Dispersions can also be prepared, for example, in glycerol, liquid polyethylene glycols, and mixtures thereof, and in oils.

The pharmaceutical forms suitable for injectable use include sterile aqueous solutions (where water soluble) or dispersions and sterile powders for the extemporaneous preparation of sterile injectable solutions or dispersions. In all cases the form must be sterile and must be fluid to the extent that easy syringability exists. It must be stable under the conditions of manufacture and storage. The carrier can be a solvent or dispersion medium containing, for example, water, DMSO, ethanol, polyol (for example, glycerol, propylene glycol, liquid polyethylene glycol, and the like), suitable mixtures thereof and vegetable oils. The proper fluidity can be maintained, for example, by the use of a coating such as lecithin, by the maintenance of the required particle size in the case of dispersion. In many cases it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the organozinc compound in the required amount in the appropriate solvent with various of the other ingredients enumerated above, as required, followed by filtered sterilization. Generally, dispersions are prepared by incorporating the various sterilized zinc ionophores into a sterile vehicle which contains the basic dispersion medium and the required other ingredients from those enumerated above. In the case of sterile powders for the preparation of sterile injectable solutions, the preferred methods of preparation are vacuum-drying and the freeze-drying technique which yields a powder of the active ingredient plus any additional desired ingredient from previously sterile-filtered solution thereof.

For oral therapeutic administration, the organozinc compound may be incorporated with excipients and used in the form of ingestible tablets, buccal tablets, troches, capsules, elixirs, suspensions, syrups, wafers, and the like. Compositions or preparations according to the present invention are prepared so that an oral dosage unit form contains a organozinc compound concentration sufficient to treat pain in a patient.

The tablets, troches, pills, capsules, and the like, may contain the following: a binder such as gum tragacanth, acacia, corn starch or gelatin; excipients such as dicalcium phosphate; a disintegrating agent such as corn starch, potato starch, alginic acid, and the like; a lubricant such as magnesium stearate; and a sweetening agent such as sucrose, lactose or saccharin may be added or a flavoring agent such as peppermint, oil or wintergreen or cherry flavoring. When the dosage unit form is a capsule, it may contain, in addition to materials of the above type, a liquid carrier. Various other materials may be present as coatings or to otherwise modify the physical form of the dosage unit. For instance, tablets, pills or capsules or zinc ionophore in suspension may be coated with shellac, sugar or both.

A syrup or elixir may contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the organozinc compound may be incorporated into sustained-release preparations and formulations.

Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragacanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water, isotonic saline; and phosphate buffer solution; skim milk powder; as well as other non-toxic compatible substances used in pharmaceutical formulations such as Vitamin C, estrogen and echinacea, for example. Wetting agents and lubricants such as sodium lauryl sulfate, as well as coloring agents, flavoring agents, lubricants, excipients, tableting agents, stabilizers, anti-oxidants and preservatives, can also be present.

The present methods for treating KCNQ related disorders, including pain, can further comprise an effective amount of another therapeutic agent. The other therapeutic agent includes, but is not limited to, an opioid agonist, a non-opioid analgesic, a non-steroid anti-inflammatory agent, an antimigraine agent, a Cox-H inhibitor, an antiemetic, a β-adrenergic blocker, an anticonvulsant, an antidepressant, a Ca²⁺-channel blocker, an anticancer agent, an agent for treating or preventing UI, an agent for treating or preventing an ulcer, an agent for treating or preventing IBD, an agent for treating or preventing IBS, an agent for treating addictive disorder, an agent for treating Parkinson's disease and parkinsonism, an agent for treating anxiety, an agent for treating epilepsy, an agent for treating a stroke, an agent for treating a seizure, an agent for treating a pruritic condition, an agent for treating psychosis, an agent for treating Huntington's chorea, an agent for treating ALS, an agent for treating a cognitive disorder, an agent for treating a migraine, an agent for treating or inhibiting vomiting, an agent for treating dyskinesia, or an agent for treating depression, and mixtures thereof.

### EXAMPLES

The present invention will now be demonstrated using specific examples that are not to be construed as limiting.

### Example 1

Cell culture and transient transfection—Chinese Hamster Ovary (CHO) cells were grown in 50/50 DMEM/F12 (Cellgro) with 10% FBS (Gibco), 100 U/ml penicillin (Cellgro), 100 μg/ml streptomycin (Cellgro) and 2 mM L-glutamine (Gibco). At 24 hours before transfection, cells were split and plated in 60 mm dishes and were transfected with Lipofectamine2000™ reagent (Invitrogen) according to the manufacturer's instruction. At 24 hours after transfection, cells were split and re-plated onto coverslips coated with poly-L-lysine (Sigma). Plasmid expressing CD4 cDNA as a marker was cotransfected with the channel cDNAs of hKCNQ1 (from Dr. Michael Sanguinetti), hKCNQ4 (from Dr. Vitya Vardanyan), rKCNQ2, rKCNQ3, and hKCNQ5 (from Drs. David McKinnon, Mark Shapiro, and Thomas Jentsch). Prior to recording, anti-CD4 Dynabeads (Dynal. Biotech. Inc.) were added into the medium to allow for identification of the transfected cells. Stable lines expressing Kv2.1, hERG, and Kv4.2 were generated by standard protocols using pcDNA3.1 and maintained at complete medium supplemented with 500 μg/ml G418. The N-type calcium channel stable line was a kind gift from Drs. Diane Lipscombe and David Yue.

### Example 2

KCNQ2 screen—HEK 293 cells stably expressing rat KCNQ2 were resuspended in DMEM/F12 medium and plated at a density of 4×10⁴/well in poly-L-Lysine coated 96-well plates. Cells were then incubated at 37° C. with 5% CO₂ overnight. 30 μl/well DMEM/F12 medium containing 30 mM RbCl was added to the cells the next day, followed by incubation for an additional 3 hours. Cell plating and reagent dispensing were done using a Multidrop 384 dispenser (Thermo Electron Corporation).

Compound addition and Rb⁺ efflux assay were programmed and performed on a Cybi-Well™ system and Tekbench liquid handling system (Tekcel Inc., Hopkinton, Mass.), respectively. Briefly, 0.9 μl/each of the 200× compound solution was added to 80 wells of each cell plate. To ensure assay reproducibility, 0.9 μl of 5% DMSO solution was added to each well in the first column of each cell plate along with the compound solutions; these wells were used as negative controls. Final DMSO concentration in the cell medium was kept at or below 0.1% for all cell plates to minimize any toxicity. After adding the compounds, the cells were again incubated at 37° C. with 5% CO₂ for 3 hours. Then each cell plate was washed twice with 200 μl/well Rb⁺ free DMEM/F12 medium and redispensed with DMEM/F12 medium containing 50 mM KCl. After incubation at room temperature for 10 minutes, the supernatant was transferred to a new 96-well plate. The cells were then lysed with 200 μl/well 1% Triton in PBS. Rb⁺ concentrations in both supernatant and cell lysates were measured by atomic absorption spectrometry using an ICR8000 instrument (Aurora Biomed, Vancouver, Canada).

### Example 3

Mutagenesis—Starting from KCNQ cDNAs, the KCNQ2 point mutants were constructed by recombinant PCR and verified by sequencing.

### Example 4

Modeling—Three-dimensional structural models for the KCNQ2 S5-S6 domains were generated using the solved crystal structure of Kv1.2 (2A79) as a template. The corresponding domains between KCNQ2 and Kv1.2 were aligned with DNASTAR MegAlign program using standard parameters. The KCNQ2 models were constructed using DeepView/SWISS-PdbViewer (http://ca.expasy.org/spdbv). The structural representation was performed with the POV-Ray program (http://www.povray.org).

### Example 5

Neuron Culture—Dorsal root ganglia (DRG) was collected from fourteen-day-old CD rat and incubated in collagenase (sigma, 500 U/ml, 15 min) and then trypsin (Sigma, 1 mg/ml, 30 min) at 37° C. Single DRG neurons were isolated by mechanical trituration with a fire-polished glass Pasteur pipette. Hippocampal tissue was dissected out from CD rat postnatal day 0. The tissue was digested with papain (Worthington Biochem, 20 U/ml, 30 min) and resuspended into single cells. The single-cell suspension was plated onto a monolayer of glial cells that were growing on coated cover slips. Cytosine-1-D-arabinofuranoside at 5 μM was added into the culture medium 24-48 hours later to arrest glial cell proliferation. The culture medium consisted of Neurobasal™ medium with B-27 supplement (Gibco), penicillin/streptomycin and 2 mM L-glutamine. All cells were maintained at 37° C. with 5% CO₂ prior to recording.

### Example 6

Electrophysiological Recording—Standard whole-cell recording was used. Pipettes were pulled from borosilicate glass capillaries (TW150-4, World Precision Instruments, Sarasota, Fla.). When filled with the intracellular solution, the pipettes have resistances of 3-5 NΩ. During the recording, constant perfusion of extracellular solution was maintained using a BPS perfusion system (ALA, Westbury, N.Y.). Pipette solution contained (mM): KCl 145, MgCl₂ 1, EGTA 5, HEPES 10, MgATP 5 (pH 7.3); extracellular solution contained (mM): NaCl 140, KCl 3, CaCl₂ 2, MgCl₂ 1.5, HEPES 10, glucose 10 (pH 7.4). Current and voltage were recorded using an Axopatch-200A amplifier, filtered at 1 kHz and digitized using a DigiData 1322A with pClamp 9.2 software (Axon Instruments, Foster City, Calif.). Series resistance compensation was also used and set to 60-80%.

For single channel recording procedure, outside-out patch recording was used. The pipettes had resistances of 7-15 MΩ when filled with the intracellular solution of the following composition (mM): KCl 145, MgCl₂ 1, EGTA 5, HEPES 10, MgATP 5 (pH 7.3). Extracellular solution contained (mM): NaCl 150, KCl 5, CaCl₂ 2, MgCl₂ 1, HEPES 10, glucose 10 (pH 7.4). Current and voltage were recorded using an Axopatch-200B amplifier, sample at 4 kHz and filtered at 200 Hz, and digitized using a DigiData 1322A with pClamp 9.2 software (Axon Instruments, Foster City, Calif.).

The open probability (Pₒ) and single channel current (i) were calculated by fitting all-point histograms with single- or multi-Gaussian curves. The ratio of the area under the fitted ‘open’ Gaussian to the total area under the entire Gaussian was taken as Pₒ, and the difference between the fitted “closed” and “open” peaks was taken as i, single channel conductance (γ) was calculated by the equation: γ=i/(V−V₅).

### Example 7

Data and Statistical Analysis—Patch-clamp data were preprocessed using Clampfit 9.2 (Axon Instruments, Foster City, Calif.) and then analyzed in Origin 7 (OriginLab, Northampton, Mass.). The activation curve was fitted by the Boltzmann equation: G=(Gₘₐₓ−Gₘᵢₙ)/[1+exp[(V−V_{1/2})/S]]+Gₘᵢₙ, where Gₘₐₓ is the maximum conductance, Gₘᵢₙ is the minimum conductance, V_{1/2} is the voltage for half of the total numbers of channels to open, and S is the slope factor. The dose response curve was fitted by the Hill equation: E=Eₘₐₓ/(1+(EC₅₀/C)^{P}), where Eₘₐₓ is the maximum response, C is the drug concentration, EC₅₀ is the drug concentration producing half of the maximum response, and P is the Hill coefficient. The deactivation trace was fitted by the standard exponential equation: I(t)=ΣIᵢ*exp(−t/τᵢ), where I is the current, t is the time and τ is the time constant. Data are presented as means±SE. Significance was estimated using paired two-tailed Student's t-test.

### Example 8

### Expression of KCNQ1 mRNA in Human Dorsal Root Ganglion

Expression of KCNQ1 mRNA is detected by PCR amplification of human dorsal root ganglion (DRG) cDNA. cDNA is prepared by reverse transcription of total RNA from human DRG using standard procedures. A second mock reverse-transcription reaction is also performed, which is identical to the first, except for the omission of the reverse transcriptase. 35 cycles of amplification are performed on a single microliter of human DRG cDNA, using oligonucleotide primers designed to amplify KCNQ1.

PCR amplified DNA fragments are separated by agarose gel electrophoresis, are visualized using ethidium bromide staining and are sized by comparison to DNA fragments of known size. PCR fails to amplify KCNQ1 fragments from reverse-transcription samples generated in the absence of reverse transcriptase (−), indicating that the DRG RNA samples are not contaminated with genomic DNA. PCR did amplify KCNQ1 from reverse transcribed cDNA samples, indicating that KCNQ2 and KCNQ3 mRNA are expressed in human dorsal root ganglion.

### Example 9

### Expression of Recombinant KCNQ1 Channel CHO Cells

A cloned KCNQ1 channel is expressed in chinese hamster ovary cells (CHO-K1 cells) according to standard methodology. CHO-K1 cells are transfected with human KCNQ1 nucleic acid using lipofectamine reagent according to the manufacturer's instructions. Cells stably expressing KCNQ1 are identified by their resistance to G418 (400 μg/ml). CHO-K1 cells stably transfected with the KCNQ1 tandem construct are maintained in Ham's F-12 supplemented with 10% heat-inactivated fetal bovine serum and 400 μg/ml G418 in an incubator at 37° C. with a humidified atmosphere of 5% CO₂.

For modulation of KCNQ channels, a benzanilide KCNQ channel opener is applied to the cells. The compound increases holding current at −40 mV and hyperpolarizes the membrane potential.

### Example 10

### Expression of Endogenous KCNQ1 Channel in DRGs

DRGs are isolated from 1 day old Sprague-Dawley rats. DRGs are dissociated using trypsin (0.25%) and protease type XXIII (2 mg/ml) and neurons are maintained in culture in 90% Eagles MEM (without L-glutamate), 10% FCS, 100 U/m; penicillin, 100 g/ml streptomycin, in an incubator at 37° C. with a humidified atmosphere of 5% CO₂.

As described above, a benzanilide KCNQ channel opener is applied to the cells. The opener increases holding current at −30 mV and hyperpolarizes the membrane potential.

### Example 11

### In Vivo Formalin Alaesia Test

The analgesic effect of a KCNQ modulator is assessed in vivo, using the formalin algesia test. All animal experiments are conducted in accordance with the Declaration of Helsinki and with the guide for the care and use of laboratory animals. In the formalin algesia test, mice are administered an IP dose of 30 mg/kg of a benzanilide KCNQ opener, or vehicle alone without opener as a control. Thirty minutes later, 20 μL of a 2.5% a formalin solution is injected into the plantar surface of the right hind paw. For thirty minutes immediately following the injection, mice are observed and the time spent licking the paw (a response to pain) is measured using a timer. Untreated mice spend more than four minutes licking the right hind paw, whereas mice treated with the KCNQ opener spend less than one minute licking the right hind paw.

### Example 12

### In Vivo Hotplate Test for Pain

In the hotplate test, mice are administered an oral dose of 10, 30, or 100 mg/kg of a benzanilide KCNQ opener. All animal experiments are conducted in accordance with the Declaration of Helsinki and with the guide for the care and use of laboratory animals. One hour later the mouse is placed on a metal surface heated to 55° C. When the mouse licks its hind paw, or after 30 seconds, it is removed from the surface, and the latency to the lick is measured. The KCNQ opener compound increases the latency to lick a hind paw. When analyzed by analysis of variance, there is an overall significant effect of compound (p<0.001), with the 30 and 100 mg/kg doses significantly different from the vehicle. Both tests show statistically significant differences between treated and untreated mice.

### Example 13

### In Vivo Geller Conflict Test for Anxiolytics

In the Geller conflict test (see, e.g., Geller & Seifter, Psychophamracologia 1:482-492 (1960: Pollard & Howard, Psychopharmacology 62:117-121 (1979)), rats are trained to press a lever to receive food pellets during daily 1 hour sessions. The sessions are divided into punished and unpunished phases. During the four, three-minute punished periods, a light signals that each lever press will produce both a pellet and a foot shock (punishment), which reduces lever pressing. The number of punished lever presses on test days (when test compound is administered) is compared to the mean on baseline days. The positive control, chlordiazepoxide, increases punished lever pressing by >50%. A compound that produces an increase of approximately 40% or greater is generally considered to be of interest as a rapid-onset anxiolytic.

A compound with selective KCNQ1 channel opening activity increases punished responding in a dose-dependent manner. The increase in punished responding is statistically significant (paired t-test p<0.05) at 10, 17, 30, and 56 mg/kg PO with increases of 40% or greater at 30 and 56 mg/kg. Responding in the unpunished phase is not disrupted, indicating that the animals are not impaired at the doses tested.

Another embodiment of the invention is a KCNQ modulator compound made by a process delineated herein, including the processes described herein. Another aspect of the invention is a KCNQ modulator compound for use in the treatment or prevention in a subject of a disease, disorder or symptom thereof delineated herein. Another aspect of the invention is use of a KCNQ modulator compound in the manufacture of a medicament for treatment or prevention in a subject of a disease, disorder or symptom thereof delineated herein.

Various modifications may be made without departing from the invention. The disclosure is to be construed as exemplary, rather than limiting, and such changes within the principles of the invention as are obvious to one skilled in the art are intended to be included within the scope of the claims.

### INCORPORATION BY REFERENCE

The contents of all references (including literature references, issued patents, published patent applications, and co pending patent applications) cited throughout this application are hereby expressly incorporated herein in their entireties by reference.

### EQUIVALENTS

Those skilled in the art will recognize, or be able to ascertain using no more than routine experimentation, many equivalents of the specific embodiments of the invention described herein. Such equivalents are intended with be encompassed by the following claims.

What is claimed:

1. A method of treating a KCNQ related disorder in a subject, comprising administering to said subject, an effective amount of a KCNQ polypeptide channel activity modulator, such that said subject is treated for said disorder;
wherein the KCNQ polypeptide channel activity modulator is an organozinc compound.

2. A method of treating a KCNQ related disorder in a subject, wherein the subject has been identified as in need of treatment for a KCNQ related disorder, comprising administering to said subject in need thereof, an effective amount of a KCNQ polypeptide channel activity modulator, such that said subject is treated for said disorder;
wherein the modulator of a KCNQ polypeptide channel is an organozinc compound.

3. The method of claim 1 or claim 2, wherein the KCNQ polypeptide channel activity modulator interacts with a KCNQ polypeptide channel.

4. The method of claim 1 or claim 2, wherein the organozinc compound is selected from zinc pyrithione (Zn-Py), zinc heterocyclic amines, zinc dithiocarbamates, and zinc vitamins.

5. The method of claim 4, wherein the organozinc compound is Zinc Pyrithione (ZnPy):

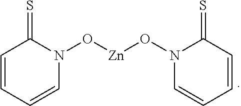

6. The method of claim 4, wherein the zinc-heterocyclic amine is selected from zinc-5,7-Diiodo-8-hydroxyquinoline and zinc-8-Hydroxyquinoline.

7. The method of claim 4, wherein the zinc-dithiocarbamates are selected from zinc-pyrrolidine dithiocarbamate, zinc-diethyldithiocarbamate, zinc-disulfiram and zinc-dimethyldithiocarbamate.

8. The method of claim 4, wherein the zinc-vitamin is selected from zinc-vitamin E and zinc-vitamin A.

9. The method of claim 1 or claim 2, wherein the organozinc compound is a zinc moiety bound, coordinated, or chelated to a compound of formula I: 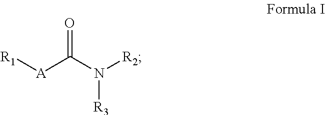
wherein:
A is a bond, CH₂, CHR_{b}, CH₂S, CHR_{b}S, CH₂O, CH₂NR_{c}, or NH;
Rb is alkyl;
R_{c} is H or S(O)ₘ-aryl;
R₁ is an optionally substituted alkyl, an optionally substituted aryl, an optionally substituted cycloalkyl, or an optionally substituted heteroaryl;
R₂ is an optionally substituted alkyl, an optionally substituted alkenyl, an optionally substituted aryl, an optionally substituted cycloalkyl, an optionally substituted heteroaryl, an optionally substituted heterocyclic, an optionally substituted aralkyl, 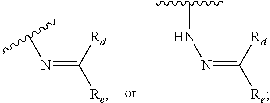
R₃ is H or alkyl;
each R_{d} and Rₑ is independently an optionally substituted alkyl, an optionally substituted aryl, or R_{d} and Rₑ together form an optionally substituted cycloalkyl; and
m is 0, 1, or 2.

10. The method of claim 1 or claim 2, wherein the organozinc compound is zinc moiety bound, coordinated, or chelated to a compound of formula II: 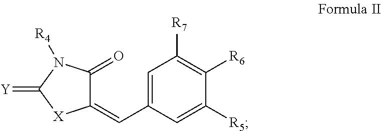
wherein:
R₄ is H, an optionally substituted alkyl, an optionally substituted alkenyl, alkynyl, allyl, or an optionally substituted aryl;
R₅ is H, hal, or hydroxyl;
R₆ is H, hal, hydroxyl, NH₂, a mono- or di-substituted amine, or an optionally substituted alkoxy;
R₇ is H, hal, hydroxyl, an optionally substituted alkoxy, or nitro;
X is S or NRₐ;
Y is O, S, or NRₐ; and
each Rₐ is independently H or an optionally substituted aryl.

11. The method of claim 1 or claim 2, wherein the organozinc compound is a zinc moiety bound, coordinated, or chelated to the following: N-Benzo[g]quinolin-4-yl-N′-(2-diethylamino-ethyl)-benzene-1,4-diamine; 2-[2-(3,4-Dihydroxy-phenyl)-2-oxo-ethylsulfanyl]-4,6-dimethyl-nicotinonitrile; 2-[2-(4-Methoxy-phenyl)-2-oxo-ethylsulfanyl]-4-(5-methyl-furan-2-yl)-5,6,7,8-tetrahydro-quinoline-3-carbonitrile; 6-Methyl-4-(5-methyl-furan-2-yl)-2-(2-oxo-2-phenyl-ethylsulfanyl)-nicotinonitrile; 2-(2-Oxo-2-thiophen-2-yl-ethylsulfanyl)-4-pyridin-4-yl-5,6,7,8-tetrahydro-quinoline-3-carbonitrile; 2-(3,5-Diiodo-2-methoxy-phenyl)-2,3,5,6,7,8-hexahydro-1H-benzo[4,5]thieno[2,3-d]pyrimidin-4-one; 2,2,2-Trifluoro-1-[1-(2,2,2-trifluoro-1-hydroxy-1-trifluoromethyl-ethyl)-1H-pyrrol-2-yl]-ethanone; 1,5-Diphenyl-1H-pyrazole-3-carboxylic acid tert-butylamide; 3-(4-Bromo-phenyl)-5-(3-phenyl-allylidene)-dihydro-pyrimidine-2,4-dione; 2-Amino-7-hydroxy-6-[(2-iodo-phenylimino)-methyl]-4-phenyl-4H-chromene-3-carbonitrile; 3-(1H-Benzoimidazol-2-yl)-6-nitro-chromen-2-ylideneamine; 6-Methoxy-3-(4-nitro-phenyl)-chroman-2-one; 2-(Benzo[1,2,5]thiadiazol-4-yliminomethyl)-benzo[b]thiophen-3-ol; 4-[3-(4-Bromo-phenyl)-3-oxo-propenylamino]-N-(4,6-dimethyl-pyrimidin-2-yl)-benzenesulfonamide; 2-[(5-Nitro-furan-2-ylmethylene)-amino]-benzamide; 2-Benzo[4,5]imidazo[1,2-c]quinazolin-6-yl-phenylamine; Dimethyl-phenyl)-5-(3-phenyl-allylidene)-pyrimidine-2,4,6-trione; 4-(4-Cyclohexyl-phenyl)-thiazol-2-ylamine; or 4-(4-Cyclohexyl-phenyl)-thiazol-2-ylamine.

12. The method of claim 1 or claim 2, wherein the KCNQ related disorder is pain, epilepsy, myokymia or cardiac disease.

13. The method of claim 12, wherein the disorder is pain.

14. The method of claim 13, wherein the pain is somatic pain.

15. The method of claim 13, wherein the pain is cutaneous.

16. The method of claim 13, wherein the pain is visceral.

17. The method of claim 13, wherein the pain is caused by a burn, a bruise, an abrasion, a laceration, a broken bone, a torn ligament, a torn tendon, a torn muscle, a viral infection, a bacterial infection, a protozoal infection, a fungal infection, contact dermatitis, inflammation, or cancer.

18. The method of claim 17, wherein the inflammation is caused by trauma, infection, surgery, burns, or diseases with an inflammatory component.

19. The method of claim 13, wherein the pain is neuropathic.

20. The method of claim 19, wherein the neuropathic pain is caused by injury to the central or peripheral nervous system due to cancer, HIV infection, tissue trauma, infection, autoimmune disease, diabetes, arthritis, diabetic neuropathy, trigeminal neuralgia or drug administration.

21. The method of claim 12, wherein the cardiac disease is long QT syndrome (LQTS), heart failure, fibrillation, or arrhythmia.

22. The method of claim 3, wherein the KCNQ channel is a heteromeric channel.

23. The method of claim 3, wherein the KCNQ channel is a homomeric channel.

24. The method of claim 3, wherein the KCNQ channel comprises at least one of KCNQ 1, KCNQ 2, KCNQ 3, KCNQ 4, and KCNQ 5.

25. The method of claim 24, wherein the KCNQ channel is KCNQ 1.

26. The method of claim 1 or claim 2, wherein the effective amount of organozinc compound ranges from about 0.005 μg/kg to about 200 mg/kg.

27. The method of claim 26, wherein the effective amount of organozinc compound ranges from about 0.1 mg/kg to about 200 mg/kg.

28. The method of claim 27, wherein the effective amount of organozinc compound ranges from about 10 mg/kg to 100 mg/kg.

29. The method of claim 1 or claim 2, wherein the organozinc compound is administered intravenously, intramuscularly, subcutaneously, intracerebroventricularly, orally or topically.

30. The method of claim 3, wherein the interaction of the organozinc compound with the KCNQ channel causes a hyperpolarization shift of voltage sensitivity of the KCNQ channel.

31. The method of claim 3, wherein the interaction of the organozinc compound with the KCNQ channel reduces the deactivation of the KCNQ channel.

32. The method of claim 3, wherein the interaction of the organozinc compound with the KCNQ channel causes a conformational change of the KCNQ channel.

33. The method of claim 32, wherein the conformational change is an opening of the KCNQ channel.

34. The method of claim 1 or claim 2, wherein the activity is inhibited or stimulated.

35. The method of claim 3, wherein the organozinc compound and the KCNQ channel interact to provide an increase or a decrease in voltage potential.

36. The method of claim 3, wherein the KCNQ channel comprises a S5 protein, a S6 protein, and a pore region.

37. The method of claim 36, wherein the organozinc compound interacts with the S5-pore-S6 protein region of the KCNQ channel.

38. The method of claim 37, wherein the interaction of the KCNQ channel with the organozinc compound results in opening of the pore and a change in voltage.

39. The method of claim 37 wherein the organozinc compound interacts with cysteine residues.

40. The method of claim 37, wherein the organozinc compound interacts with alanine residues.

41. The method of claim 37, wherein the organozinc compound interacts with Trp and Gly residues.

42. The method of claim 3, wherein the organozinc compound and the KCNQ channel interaction takes place in a cell.

43. The method of claim 42, wherein said cell is in a mammal.

44. The method of claim 42, wherein said cell is from a mammal.

45. The method of claim 43 or claim 44, wherein said mammal is a human or a rodent.

46. The method of claim 44, wherein said cell is in vitro.

47. The method of claim 1 or claim 2 wherein the subject is a mammal.

48. The method of claim 47 wherein the subject is a primate or human.

49. The method of claim 1 or claim 2, wherein the organozinc compound is administered alone or in combination with one or more other pain therapeutics.

50. A method of protecting against the effects of pain comprising, administering to a subject in need thereof, a pharmaceutically effective amount of a KCNQ polypeptide channel activity modulator;
wherein the modulator is an organozinc compound.

51. A method of modulating the activity of a potassium channel in a subject, comprising contacting a potassium channel with an organozinc compound.

52. A method for identifying a compound which modulates the activity of a KCNQ polypeptide channel, the method comprising:
a) contacting a KCNQ polypeptide channel with a compound under conditions suitable for modulation of the activity of the KCNQ polypeptide channel; and
b) detecting modulation of the activity of the KCNQ polypeptide channel by the compound;
wherein the compound chemically interacts with the KCNQ polypeptide channel.

53. The method of claim 52, wherein the KCNQ polypeptide channel is KCNQ 1.

54. The method of claim 52, wherein the interaction of the compound with the KCNQ polypeptide channel is a binding interaction.

55. The method of claim 54, wherein the binding interaction is ionic, covalent, or a non-direct interaction.

56. The method of claim 52, further comprising the step of testing the compound for biological activity.

57. The method of claim 52, wherein the compound is identified using a rubidium efflux assay.

58. The method of claim 57, wherein the rubidium efflux assay is non-radioactive.

59. A method of treating a KCNQ mutation disorder in a subject, comprising administering to the subject, an effective amount of a KCNQ polypeptide channel activity potentiator, such that said subject is treated for said disorder,
wherein the potentiator is an organozinc compound.

60. A method of treating a KCNQ mutation disorder in a subject, wherein the subject has been identified as in need of treatment for a KCNQ mutation disorder, comprising administering to said subject in need thereof, an effective amount of a KCNQ polypeptide channel activity potentiator, such that said subject is treated for said disorder;
wherein the potentiator is an organozinc compound.
